(19) United States
(12) Patent Application Publication
(10) Pub. No.: US 2019/0230724 A1
(43) Pub. Date: Jul. 25, 2019

(54) RADIOGRAPHIC SYSTEM AND RADIOGRAPHIC COMMUNICATION SYSTEM

(71) Applicant: Konica Minolta, Inc., Tokyo (JP)

(72) Inventors: Masahiro KUWATA, Tokyo (JP); Satoshi KOMIYA, Tokyo (JP); Takeshi NUKANOBU, Tokyo (JP); Tetsuo KUBOTA, Tokyo (JP); Kentaro HARA, Tokyo (JP)

(21) Appl. No.: 16/234,822

(22) Filed: Dec. 28, 2018

(30) Foreign Application Priority Data
Jan. 24, 2018 (JP) 2018-009975

(51) Int. Cl. H04W 76/11; G16H 40/67; G01T 1/17

(52) U.S. Cl. CPC H04W 76/11; G01T 1/17; G16H 40/67

(57) ABSTRACT

A radiographic communication system includes a radiographic device and an information processor. The radiographic device receives radiation to generate data of a radiographic image and that operates as a primary device in wireless communication. The information processor operates as a secondary device in the wireless communication. The information processor includes a memory, an operation interface, a communicator and a hardware processor. The memory stores combination information including a communication identifier and an address of the radiographic device. The operation interface is associated with the combination information. The hardware processor uses the communicator to establish communication with the radiographic device based on the communication identifier in the combination information in response to an operation of the operation interface. The hardware processor performs transmission and reception of data to and from the radiographic device via the communicator based on the address in the combination information.

### BACKGROUND

### 1. Technological Field

The present invention relates to a radiographic system and a radiographic communication system.

### 2. Description of the Related Art

Radiographic systems include an irradiator generating radiation, a radiographic device receiving radiation to generate data of radiographic images, and an information processor transmitting and receiving data to/from the radiographic device. A typical radiographic system includes multiple radiographic devices for one information processor for wireless communication between the information processor and the radiographic devices.

A technique for such a radiographic system is disclosed in, for example, Japanese Patent Application Publication No. 2017-6427. This technique involves defining one of the radiographic devices as a primary wireless device and the remaining radiographic devices as secondary wireless devices and transmitting image data generated by the secondary wireless devices to the information processor via the primary wireless device.

However, the technique disclosed in Japanese Patent Application Publication No. 2017-6427 has drawbacks in stability in communication:

(i) the communication load of the primary wireless device is high, thus communication failures are more likely to occur, such as a low communication rate or failed communication, and

(ii) since the communication load of the primary wireless device is high, the primary wireless device consumes more power than the secondary wireless devices, and the battery of the primary wireless device is exhausted before the secondary wireless devices and thus the period of time in which the primary wireless device can communicate is short.

In the technique disclosed in Japanese Patent Application Publication No. 2017-6427, the secondary wireless devices are in communication via the primary wireless device. If the battery of the primary wireless device is exhausted to such a degree that the primary wireless device cannot communicate, the wireless communication may be failed even if the secondary wireless devices still have live batteries. That is, the radiographic devices used as secondary wireless devices still have electric power in their batteries and can be used whereas the primary wireless device has exhausted power in its battery and cannot communicate with the information processor. This may consequently cause a risk that all the radiographic devices cannot be used.

This is crucial in fields of, for example, emergency medical services that require high mobility of radiographic devices.

### SUMMARY

An object of the present invention is to certainly establish communication between an information processor and a radiographic device capable of receiving radiation and generating data of radiographic images even if multiple radiographic devices are in use.

To achieve at least one of the abovementioned objects, according to a first aspect of the present invention, a radiographic communication system includes:

at least one radiographic device that receives radiation to generate data of a radiographic image and that operates as a primary device in wireless communication; and an information processor that operates as a secondary device in the wireless communication, wherein the information processor includes:

a memory that stores combination information including a communication identifier and an address of the radiographic device;

at least one operation interface that is associated with the combination information and is operated by a user;

a communicator; and

a hardware processor that uses the communicator to establish communication with the radiographic device based on the communication identifier in the combination information in response to an operation of the operation interface, the hardware processor performing at least one of transmission and reception of data to and from the radiographic device via the communicator based on the address in the combination information.

According to a second aspect of the present invention, a radiographic communication system includes:

a radiographic device that receives radiation to generate data of a radiographic image and that operates as a primary device in wireless communication; and

an information processor that operates as a secondary device in the wireless communication,

wherein the information processor includes:

a memory that stores combination information including a communication identifier and an address of the radiographic device, the communication identifier and the address being associated with a predetermined radiographic condition;

an operation interface that is associated with the radiographic condition and is operated by a user;

a communicator; and

a hardware processor that uses the communicator to establish communication with the radiographic device based on the communication identifier in the combination information associated with the radiographic condition selected in response to an operation of the operation interface, the hardware processor performing at least one of transmission and reception of data to and from the radiographic device based on the address in the combination information associated with the selected radiographic condition.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram illustrating a configuration of a radiographic system according to an embodiment of the present invention.

FIG. 2 is a block diagram illustrating a specific configuration of a radiographic device of the radiographic system in FIG. 1.

FIG. 3 is a block diagram illustrating a specific configuration of an information processor of the radiographic system in FIG. 1.

FIG. 4 is a schematic view illustrating usage of a radiographic communication system of the radiographic system in FIG. 1.

FIG. 5 is another schematic view illustrating usage of the radiographic communication system of the radiographic system in FIG. 1.

FIG. 6 is another schematic view illustrating usage of the radiographic communication system of the radiographic system in FIG. 1.

FIG. 7 is another schematic view illustrating usage of the radiographic communication system of the radiographic system in FIG. 1.

FIG. 8 is another schematic view illustrating usage of the radiographic communication system of the radiographic system in FIG. 1.

FIG. 9 is a table indicating the correspondence between radiographic conditions and pieces of combination information.

FIG. 10 is another table indicating the correspondence between the radiographic conditions and the combination information.

FIG. 11 is a schematic view illustrating usage of the radiographic communication system of the radiographic system in FIG. 1.

FIG. 12 is another schematic view illustrating usage of the radiographic communication system of the radiographic system in FIG. 1.

FIG. 13 is still another schematic view illustrating usage of the radiographic communication system of the radiographic system in FIG. 1.

FIG. 14 is a table indicating the correspondence between the radiographic conditions and communication identifiers.

FIG. 15 is a schematic view illustrating usage of the radiographic communication system of the radiographic system in FIG. 1.

FIG. 16 is a table indicating the correspondence between the radiographic conditions and the communication identifiers.

FIG. 17 is a schematic view illustrating usage of the radiographic communication system of the radiographic system in FIG. 1.

FIG. 18 is a block diagram of a configuration of the radiographic system according to an example of the present embodiment.

FIG. 19 is a block diagram illustrating part of the operation of the radiographic system in FIG. 18.

FIG. 20 is another block diagram illustrating part of the operation of the radiographic system in FIG. 18.

FIG. 21A is a table showing cradle identifiers stored in one console of the radiographic system in FIG. 18.

FIG. 21B is a table showing cradle identifiers stored in another console of the radiographic system in FIG. 18.

FIG. 22 is a block diagram illustrating part of the operation of the radiographic system in FIG. 18.

FIG. 23 is another block diagram illustrating part of the operation of the radiographic system in FIG. 18.

FIG. 24 is another block diagram illustrating part of the operation of the radiographic system in FIG. 18.

FIG. 25 is a conceptual diagram explaining update of the cradle identifiers in FIGS. 21A and 21B.

FIG. 26 is a block diagram illustrating part of the operation of the radiographic system in FIG. 18.

FIG. 27 is a ladder diagram of the partial operation of the radiographic system in FIG. 18.

FIG. 28 is another ladder diagram of the partial operation of the radiographic system in FIG. 18.

FIG. 29 is another ladder diagram of the partial operation of the radiographic system in FIG. 18.

FIG. 30 is another ladder diagram of the partial operation of the radiographic system in FIG. 18.

FIG. 31 is still another ladder diagram of the partial operation of the radiographic system in FIG. 18.

FIG. 32 is a conceptual diagram explaining the partial operation of the radiographic system in FIG. 18.

FIG. 33 is a schematic view illustrating a display of the console of the radiographic system in FIG. 18.

FIG. 34 is another schematic view illustrating the display of the console of the radiographic system in FIG. 18.

FIG. 35 is a schematic view of the radiographic system in FIG. 18.

FIG. 36 is another schematic view of the radiographic system in FIG. 18.

FIG. 37 is a perspective view of a radiographic device of the radiographic system in FIG. 18.

FIG. 38A is a timing diagram illustrating the operation of the radiographic device of the radiographic system in FIG. 18.

FIG. 38B is another timing diagram illustrating the operation of the radiographic device of the radiographic system in FIG. 18.

FIG. 39A is a side view of a traditional radiographic system.

FIG. 39B is a block diagram of a configuration of the traditional radiographic system.

FIG. 40A is a side view of the radiographic system according to an example of the present embodiment.

FIG. 40B is a block diagram of a configuration of the radiographic system according to an example of the embodiment.

### DETAILED DESCRIPTION OF EMBODIMENTS

Hereinafter, one or more embodiments of the present invention will be described with reference to the drawings. However, the scope of the invention is not limited to the disclosed embodiments.

Radiographic System

The outline of a radiographic system according to the present embodiment (hereinafter referred to as “radiographic system 100”) will now be described. FIG. 1 is a block diagram of the schematic configuration of the radiographic system 100.

As illustrated in FIG. 1, the radiographic system 100 according to the embodiment includes an irradiating device (hereinafter referred to as “irradiator 1”), a radiographic communication system (hereinafter referred to as “communication system S”), and a console 2.

The irradiator 1 that can generate radiation includes, for example, a generator 11 and an X-ray tube 12 (a bulb tube or radiation source).

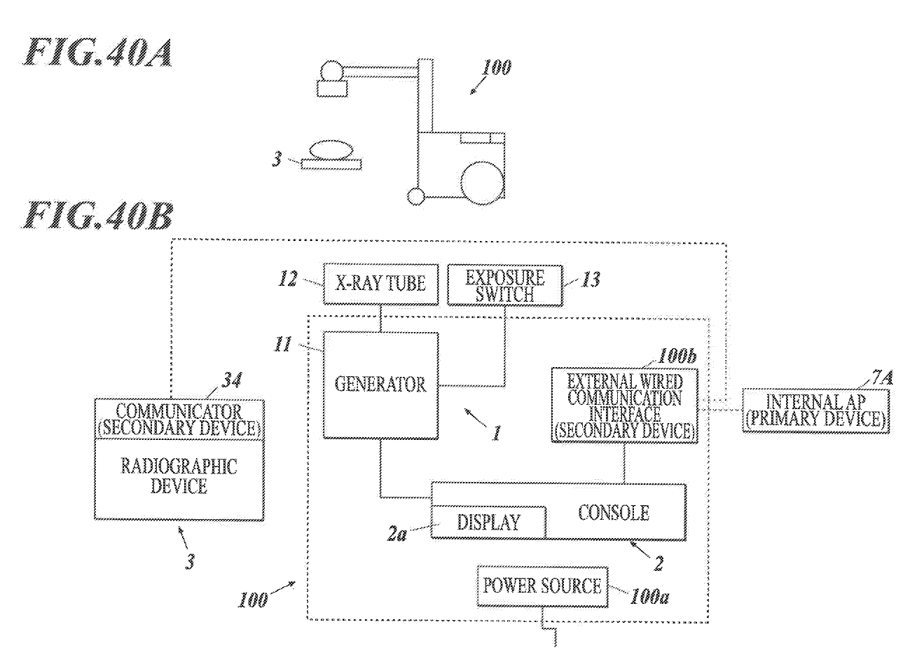

The generator 11 can apply voltages to the X-ray tube 12 under predetermined irradiating conditions (for example, tube voltage, tube current, and irradiation time (mAs values)) in response to the operation of an exposure switch 13 (see FIG. 40).

The X-ray tube 12 includes, for example, a rotary anode and a filament, which are not shown. After voltages are applied to the filament from the generator 11, the filament radiates electron beams corresponding to the applied voltage toward the rotary anode. The rotary anode in turn generates a dose of radiation X in response to the intensity of an electron beam.

The communication system S includes a radiographic device 3 and an information processor 4.

The radiographic device 3 can receive radiation to generate the data of radiographic images and wirelessly communicate with the information processor 4 as required.

The radiographic device 3 can receive various data from the information processor 4, generate the data of radiographic images corresponding to the radiation from the exterior, and transmit the data to the console 2 and the information processor 4.

The radiographic device 3 will be described in detail below.

The information processor 4 includes a mobile terminal or a dedicated device and can be in wireless communication with, for example, the radiographic device 3.

The information processor 4 can transmit various data to the radiographic device 3 and receive radiographic images from the radiographic device 3.

The information processor 4 will also be described in detail below.

The radiographic system 100 according to the present embodiment having such a configuration allows the irradiator 1 to irradiate a subject in front of the radiographic device 3 for radiography of the subject.

The image data generated by the radiographic device 3 can be transmitted to the console 2 and the information processor 4.

The radiographic system 100 according to the present embodiment having such a configuration may be disposed in, for example, a laboratory in an establishment (hospital), or may be a movable system including a movable irradiator 1 that is wheeled (hereinafter referred to as “visiting cart”).

Radiographic Device

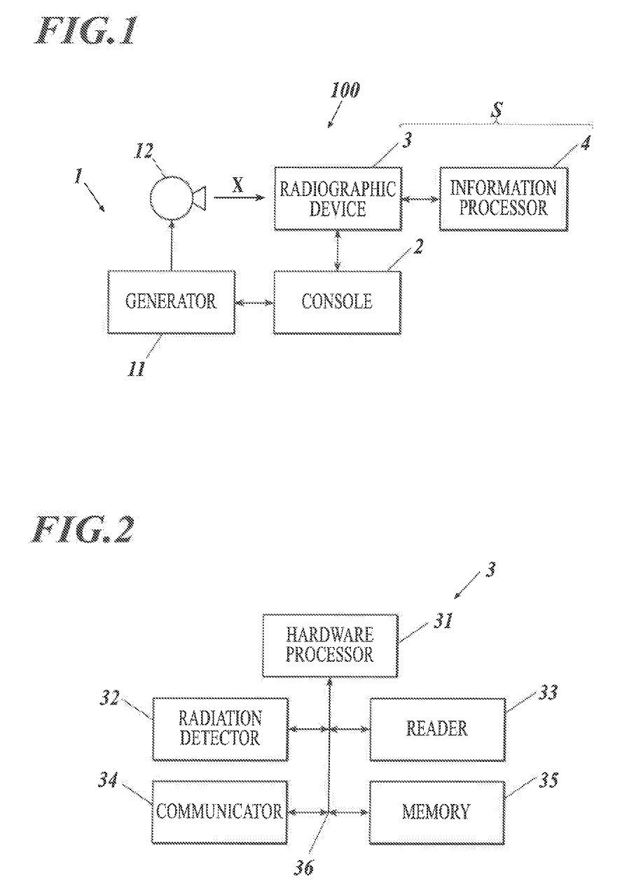

The radiographic device 3 of the radiographic system 100 will now be described in detail. FIG. 2 is a block diagram illustrating a specific configuration of the radiographic device 3.

As illustrated in FIG. 2, the radiographic device 3 includes, for example, a hardware processor 31, a radiation detector 32, a reader 33, a communicator 34, and a memory 35. The components 31 to 35 are connected via a bus 36. A built-in battery or an external power source is configured to supply power to the components 31 to 35.

The hardware processor 31 is configured to integrally control the operations of the components of the radiographic device 3 by, for example, a central processing unit (CPU) and random access memory (RAM). Specifically, the hardware processor 31 reads various processing programs stored in the memory 35 and loads the programs in the RAM to execute various processes according to the processing program in response to, for example, turning on of a power switch, reception of predetermined control signals from the irradiator 1, the console 2, and the information processor 4, and reception of radiation from the irradiator 1.

The radiation detector 32 may be any well-known traditional radiation detector that includes a plurality of pixels two-dimensionally arrayed on a substrate, each pixel including a radiation detecting element and a switch element. The radiation detecting element directly or indirectly generates an electric charge in a quantity corresponding to the dose of radiation. The switching element is disposed between the radiation detecting element and a trace, can be switched between an ON state where current can be conducted between the radiation detecting element and the trace and an OFF state where current cannot be conducted between the radiation detecting element and the trace.

In other words, the radiographic device 3 may be of a so-called indirect type including a scintillator that receives radiation and emits light to be detected, or may be of a so-called direct type that directly detects radiation without a scintillator.

The reader 33 may be any well-known traditional reader that can read the quantity of electric charge stored in each radiation detecting element as a signal value.

The communicator 34 includes, for example, a network interface and transmits/receives data to/from an external device connected via a communication network, such as local area network (LAN), wide area network (WAN), or the Internet.

The memory 35 includes, for example, a hard disk drive (HDD) or semiconductor memory and stores various processing programs including image processing programs and parameters/files necessary for execution of the programs.

The memory 35 can store its communication identifier and address.

The communication identifier is preferably the identifier of a wireless communicator (BSSID) or at least one of the identifier and access key (password) of a wireless network (ESSID).

The address is preferably at least one of a MAC address, an IP address, and a host name.

The hardware processor 31, having such a configuration, of the radiographic device 3 has the following functions:

For example, the hardware processor 31 turns off the switching element of the radiation detector 32 to store electric charge in the radiation detecting element or turns on the switching element to release the electric charge stored in the radiation detecting element to the reader 33.

The hardware processor 31 generates image data based on a signal value read by the reader 33.

The hardware processor 31 uses the communicator 34 to receive various pieces of information and signals from an external device (for example, the console 2 or the information processor 4).

The hardware processor 31 uses the communicator 34 to transmit various pieces of information and signals to an external device (for example, the console 2 or the information processor 4).

The hardware processor 31 uses the communicator 34 to operate as a primary device (hereinafter referred to as “primary wireless device”) in the wireless communication.

In the present embodiment, the hardware processor 31 may operate as a secondary device (hereinafter referred to as “secondary wireless device”) in the wireless communication and may be switched between the mode of the primary wireless device and the mode of the secondary wireless device.

The memory 35 can store image data generated by the radiation detector 32 and the reader 33.

Immediately after an image is taken, the hardware processor 31 can instruct the generated image data to be transmitted to an external device (for example, the console 2 or the information processor 4) in a wireless or wired manner (mode A).

Alternatively, the hardware processor 31 can cause the generated image data to be stored in the memory 35 and instruct the image data to be transmitted to the external device (for example, the console 2 or the information processor 4) in a wireless or wired manner in response to an instruction from the external device, an input operation through a button on the radiographic device 3, or a wireless or wired connection (mode B).

The communication system S may include multiple radiographic devices 3 each having the configuration described above and a different communication identifier and address from each other.

Information Processor

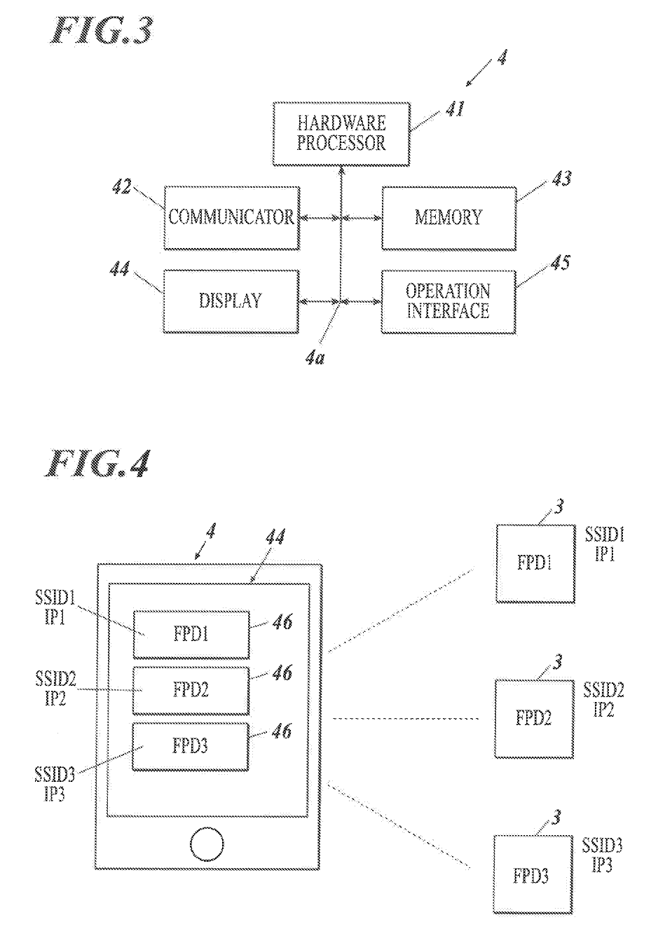

The information processor 4 of the radiographic system 100 will now be described in detail. FIG. 3 is a block diagram of a specific configuration of the information processor 4.

As illustrated in FIG. 3, the information processor 4 includes, for example, a hardware processor 41, a communicator 42, a memory 43, a display 44, and an operation interface 45. The components 41 to 45 are connected via a bus 4a.

The hardware processor 41 is configured to integrally control the operations of the components of the information processor 4 by, for example, a CPU or RAM. Specifically, the hardware processor 41 causes various processing programs stored in the memory 43 to be read and copied in the RAM and instructs various processes to be executed in accordance with the processing programs and controls displayed content on the display 44 in response to input operation signals from the operation interface 45 and reception of various signals and data from the radiographic device 3.

The communicator 42 includes, for example, a network interface and transmits and receives data to/from an external device connected through a communication network, such as LAN, WAN, or the Internet.

It should be noted that the communicator 42 may be in wireless communication with the external device via, for example, a wireless LAN or a mobile phone network for transmission and reception of data.

The memory 43 includes, for example, a hard disk drive (HDD) or a semiconductor memory and stores various processing programs and parameters/files necessary for execution of the programs.

The memory 43 stores combination information including the communication identifier and the address of the radiographic device 3.

In some cases, the memory 43 stores multiple pieces of combination information including communication identifiers and addresses corresponding to multiple radiographic devices.

The display 44 includes a monitor, such as a liquid crystal display (LCD) and displays various screens in response to instructions by display signals from the hardware processor 41.

The operation interface 45 may include one or more mechanical buttons. Alternatively, the operation interface 45 may include, for example, an icon appearing on the display 44 and operable by clicking with a mouse or touching on a touch panel on the display 44.

The operation interface 45 is associated with the combination information stored in the memory 43 and is operable by a user.

The operation interface 45 transmits operation signals in response to an operation by the user to the hardware processor 41.

The hardware processor 41, having such a configuration, of the information processor 4 has the following functions:

For example, the hardware processor 41 uses the communicator 42 to operate as a secondary wireless device.

The hardware processor 41 uses the communicator 42 to establish communication with the radiographic device 3 based on a communication identifier included in the combination information stored in the memory 43.

The hardware processor 41 uses communicator 42 to perform at least one of transmission and reception of various data to/from the radiographic device 3 based on an address included in the combination information.

Scheme of Setting of Communication Identifier and Address (I)

The description of the functions of the information processor 4 will continue. In the accompanying drawings that are referred to, multiple radiographic devices 3 are sometimes denoted as “flat panel detectors (FPD) 1 to 3”.

Based on the operation on the operation interface 45, the hardware processor 41 specifies the combination information including the corresponding communication identifier and address for the communication using the communicator 42.

In particular, if multiple pieces of combination information including communication identifiers and addresses corresponding to multiple radiographic devices are stored in the memory 43, the hardware processor specifies any of the pieces of combination information based on the operation on the operation interface 45.

Specifically, an application reading and specifying combination information stored in the memory 43 after the activation is installed in, for example, the information processor 4.

In the case that multiple radiographic devices 3 potentially communicate as primary wireless devices, applications respectively reading and specifying pieces of combination information corresponding to the radiographic devices 3 defined as primary wireless devices are installed.

The applications described above uses communication identifiers in the pieces of combination information corresponding to the radiographic devices 3 defined as primary wireless devices to establish commutation, uses addresses in the pieces of combination information to allow transmission and reception of the data to/from the primary wireless devices, and switches, for example, settings of the radiographic devices 3 and radiographic conditions.

Alternatively, the applications uses the communication identifiers in the pieces of combination information corresponding to the radiographic devices 3 defined as the primary wireless devices to establish communication, uses addresses in the combination information to allow transmission and reception of the data to/from the primary wireless devices, and acquires, for example, the number of images stored in the radiographic device 3, the storage time, and the stored image data for display of such data on the display 44 of the information processor 4.

In other words, icons 46 of the number corresponding to that of applications installed appear on the display 44 of the information processor 4 as illustrated in FIG. 4. Any of the icons 46 can be selected and clicked by a user for activation of an application, use the communication identifier in the combination information assigned to the selected application to establish communication, and use the address in the combination information to allow transmission and reception of the data to/from the primary wireless device.

For example, in FIG. 4, the icon 46 that is on the top of the application and corresponds to a FPD 1 is clicked, and then an application 47 corresponding to the FPD 1 is activated thereby as illustrated in FIG. 5.

The application 47 corresponding to the FPD 1 stores a communication identifier SSID 1 and an address IP 1 of the FPD 1 defined as a primary wireless device. The communication identifier SSID 1 can be used for establishment of the communication with the FPD 1, and the address IP 1 can be used for transmission and reception of the data to/from the FPD 1.

The application 47 allows transmission and reception of the data to/from the FPD 1 to display the radiographic conditions and settings of the FPD 1, for example, a storage time after an image is taken and a reset time (not shown). The radiographic conditions and settings can be modified on the application. The application thereby allows transmission and reception of the data to/from the FPD 1 properly and modifies the radiographic conditions and settings of the FPD 1.

The application 47 allows transmission and reception of the data to/from the FPD 1 to acquire and display the state of the FPD 1, for example, the number of images taken and stored in the memory 35 of the FPD 1, the memory space left in the FPD 1, the remaining number of images that can be taken, the remaining capacity of the battery, the state of the establishment of communication, the state of the wireless communication (not shown).

The application 47 allows transmission and reception of the data to/from the FPD 1 to acquire and display radiographic data stored in the memory 35 of the FPD 1 (at least part of, for example, the image data, the radiographic conditions, and the time when the image is taken), which is not shown.

The application 47 allows transmission and reception of the data to/from the FPD 1 to acquire the radiographic data stored in the memory 35 of the FPD 1 (at least part of, for example, the image data, the radiographic conditions, and the time when the image is taken), which is not shown, and can store the radiographic data in the memory 43 of the information processor 4. Furthermore, the application 47 can delete the radiographic data stored in the memory 35 of the FPD 1.

Scheme of Setting of Communication Identifier and Address (II)

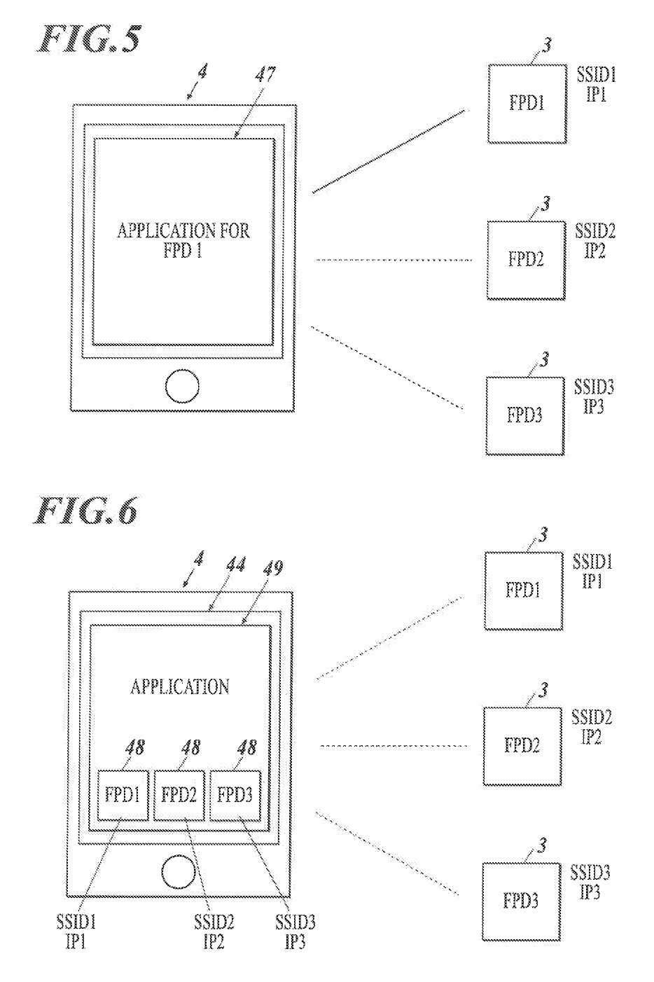

In an alternative scheme, an application 49 with selection buttons 48 corresponding to the radiographic devices 3 defined as primary wireless devices is installed in the information processor 4 as illustrated in FIG. 6.

The selection buttons 48 correspond to respective combinations of stored communication identifiers and addresses of the radiographic devices defined as primary wireless devices.

As illustrated in FIG. 7, after an selection button 48 is operated, the application may use a communication identifier in the combination information to establish the communication and use an address in the combination information to allow transmission and reception of the data to/from the primary wireless device and modify, for example, the setting and radiographic condition of the radiographic device.

For example, in FIG. 7, the selection button 48 corresponding to a FPD 2 in the center of the lower portion of the application 49 is clicked, and then the application 49 can use a stored communication identifier SSID 2 that corresponds to the selection button 48 for the FPD 2 defined as a primary wireless device to establish the communication with the FPD 2 and use an information address IP 2 to allow transmission and reception of the data to/from the FPD 2.

In the case that the applications 47 described above are used, selection of an application program corresponding to one radiographic device 3 to be used from the applications 47 is required when the radiographic device 3 defined as the primary wireless device is selected for communication, in other words, for use.

Such selection of an application sometimes requires procedures, such as termination of the application 47 corresponding to one radiographic device 3 and activation of another application 47 corresponding to another radiographic device 3, which is a troublesome and time-consuming operation. Such a troublesome and time-consuming operation is not desired in, for example, fields of emergency medical services that require quick actions.

Such fields may require the mode of the application 49 where a radiographic device to be defined as the primary wireless device can be selected on one application.

In FIGS. 4, 6, and 7, the icons 46 or the selection buttons 48 disposed on the display 44 serve as the operation interface 45. Alternatively, the operation interface may be disposed external to the display 44 and physically operated.

In the case of multiple radiographic devices 3 to be defined as primary wireless devices, one radiographic device should be selected as the primary wireless device from the radiographic devices 3 for establishment of the communication. However, in the case that combinations of communication settings for connection with multiple primary wireless devices are stored and one primary wireless device is specified from the combinations, an error in selection may occur, resulting in a connection to the untargeted radiographic device 3.

The radiography without being aware of this situation results in unintended capture of an image and an unnecessary exposure of a subject to radiation.

Advantageously, use of the schemes (I) and (II) of setting of the communication identifier and address allow display of the combinations in a visible manner and the combination can be specified based on the selection by a user. Thus, unintended setting of the communication can be reduced.

Scheme (III) of Setting of Communication Identifier and Address

An application reading and specifying the combination information stored in the memory 43 after activation is installed in the information processor 4 and is associated with a radiographic condition. In the case of multiple radiographic conditions, applications reading and specifying pieces of combination information corresponding to the respective radiographic conditions are installed.

The radiographic conditions can be, for example, the radiographic portion, the radiographic location, the radiographic room, the radiographic direction, the type of radiographic device.

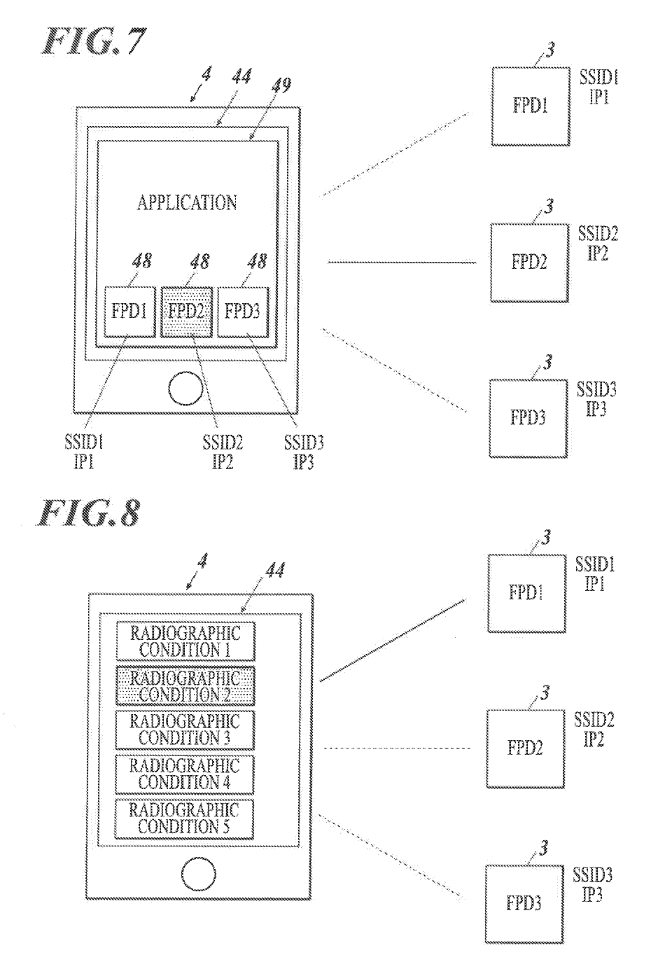

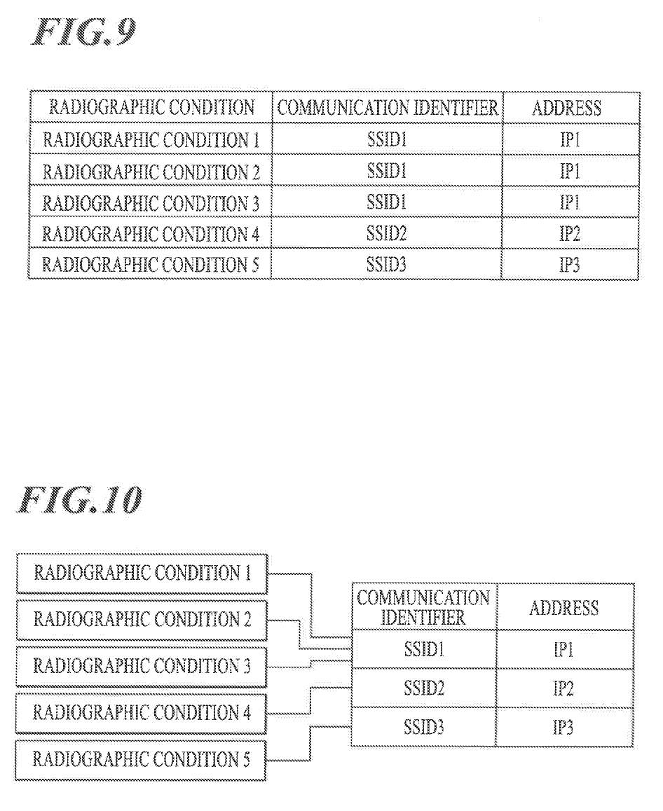

For example, in the case illustrated in FIG. 8, the display 44 of the information processor 4 displays the activation icons 46 of the application storing the combinations of communication identifiers and addresses in FIG. 9.

For example, if the icon 46 for the radiographic condition 2 is operated to activate the application corresponding to the radiographic condition 2, the communication identifier SSID 1 and the address IP 1 of the FPD 1 stored in the application corresponding to the radiographic condition 2 in FIG. 8 are used to establish communication with the FPD 1 and allows transmission and reception of the data to/from the FPD 1.

As a result, after transmission and reception of the data to/from the FPD 1 is allowed, the application 47 corresponding to the FPD 1 in FIG. 5 is activated and allows, for example, setting of the FPD 1, modification of the radiographic condition, display of the state of the FPD 1, display of an image taken by the FPD 1.

Scheme (IV) of Setting of Communication Identifier and Address

The application 49 can store one or more other radiographic conditions. As illustrated in FIG. 10, a combination of a communication identifier and an address that corresponds to a radiographic condition can be stored.

As illustrated in FIG. 11, the application 49 on the display 44 of the information processor 4 can be provided with the selection buttons 48 for respective radiographic conditions on the top. After the selection button 48 of the application 49 is operated, the application 49 uses a combination of a communication identifier and an address associated with a radiographic condition stored in the memory 43 as illustrated in FIG. 10 to establish the communication and allows transmission and reception of the data.

For example, in the case illustrated in FIG. 11, the selection button 48 for a radiographic condition 5 is selected on the application 49. The application 49 then uses a communication identifier SSID 3 corresponding to the radiographic condition 5 based on the associated information on the radiographic condition, the communication identifier, and the address stored in memory 43 as illustrated in FIG. 10 and establishes the communication. The application 49 uses an address IP 3 corresponding to the radiographic condition 5 to set the communicator to transmit and receive the data. As a result, the transmission and reception of the data to/from the FPD 3 corresponding to the SSID 3 and IP 3 is allowed.

In this manner, the schemes (III) and (IV) of setting of the communication identifier and the address are used and the radiographic condition is selected by the user. The communication with the radiographic device 3 defined as the primary wireless device is thereby allowed. Thus, the radiographic device 3 can be readily connected to the information processor 4.

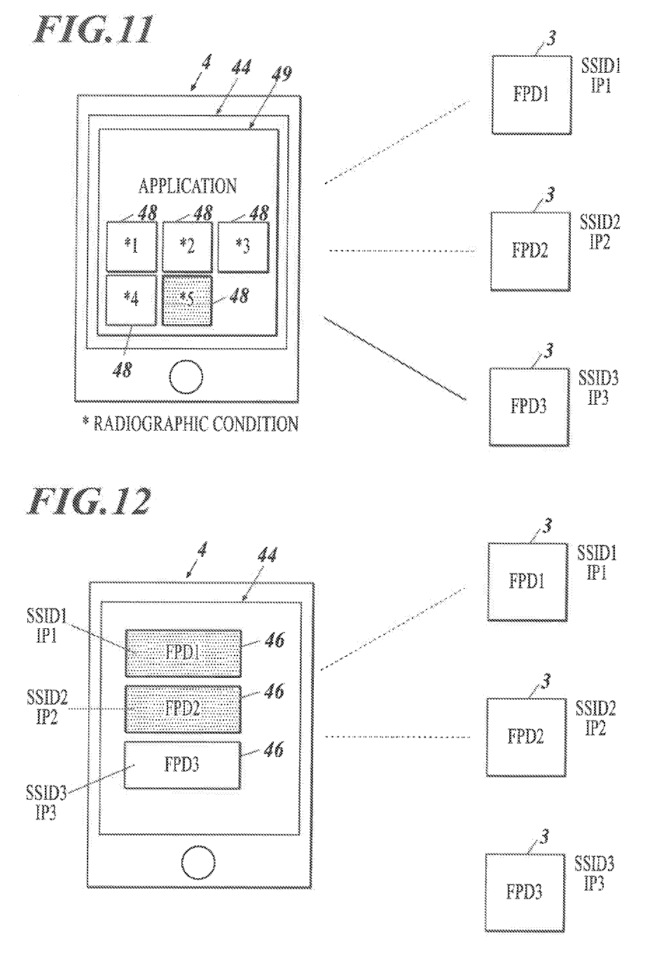

In FIGS. 4 to 8 and 11 that have been described and in FIGS. 12, 13, 15, and 17 that will be described below, the straight solid lines and dotted lines indicate the state of the communication between the information processor 4 and the radiographic device 3. The solid lines indicate that the communication between the information processor 4 and the radiographic device 3 is established and the data can be transmitted and received.

The dotted lines indicate that the information processor 4 can receive the electric waves from the radiographic device 3 defined as the primary wireless device and that the data can be transmitted or received after the procedures for establishment of the communication.

For example, in general settings of wireless communication, the communicator 42 of the information processor 4 can detect the SSID of the radiographic device 3 defined as the primary wireless device connected in the state represented by the dotted lines.

Display (I) of Connectable Radiographic Device

As illustrated in FIGS. 12 and 13, the icon 46 or the selection button 48 corresponding to the SSID detectable by the information processor 4 is displayed in a mode different from that of the icon 46 or the selection button 48 corresponding to the SSID undetectable by the information processor 4. This can inform the user of which radiographic device 3 can be in communication.

Display (II) of Connectable Radiographic Device

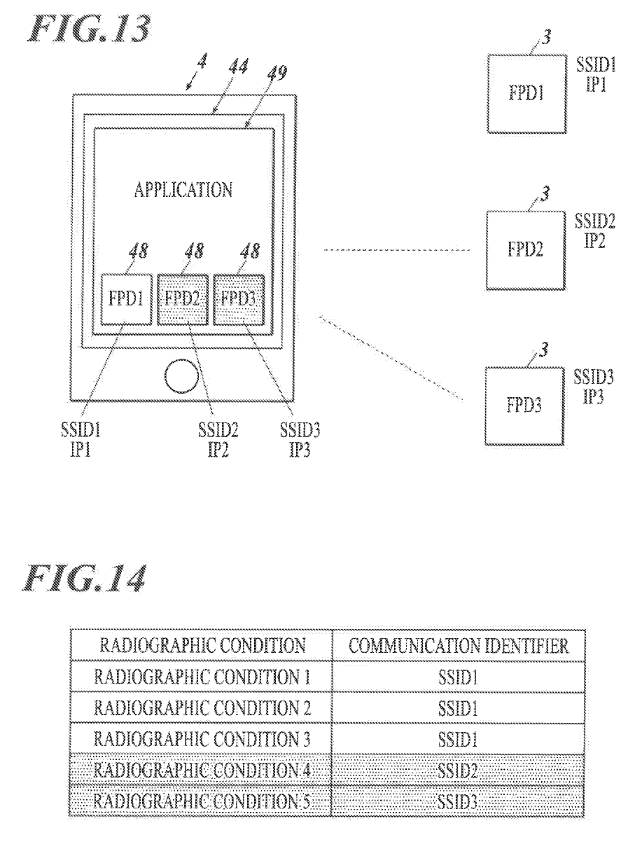

In a similar manner, the radiographic condition corresponding to the SSID detectable by the information processor 4 can be determined from the associated information on the communication identifiers and the radiographic conditions stored in the memory 43 of the information processor 4 as illustrated in FIG. 14. As illustrated in FIG. 15, the radiographic condition corresponding to the SSID detectable by the information processor 4 is displayed in a mode different from that of the radiographic condition corresponding to the SSID undetectable by the information processor 4. This can inform the user of which radiographic condition is usable.

Display (III) of Connectable Radiographic Device

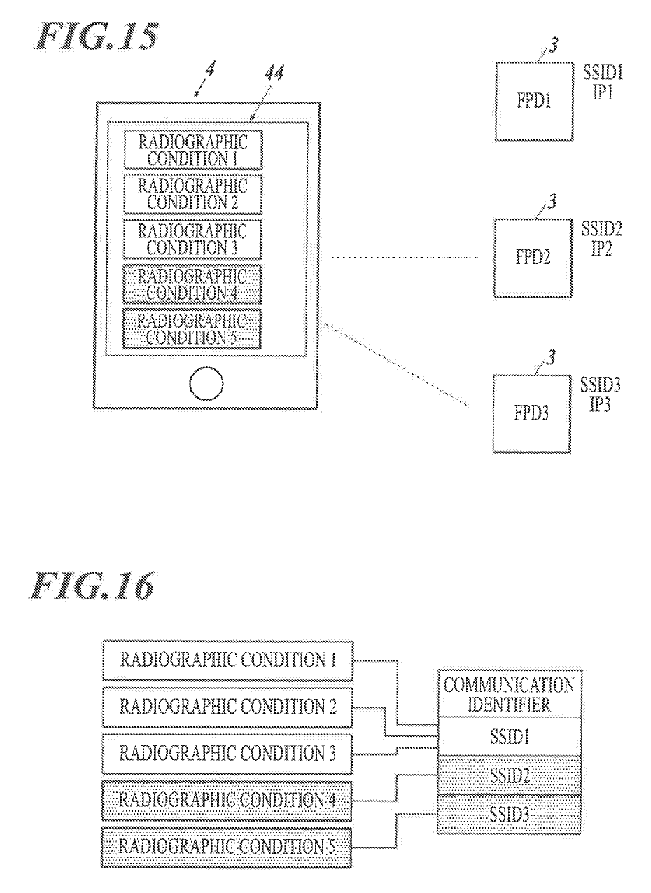

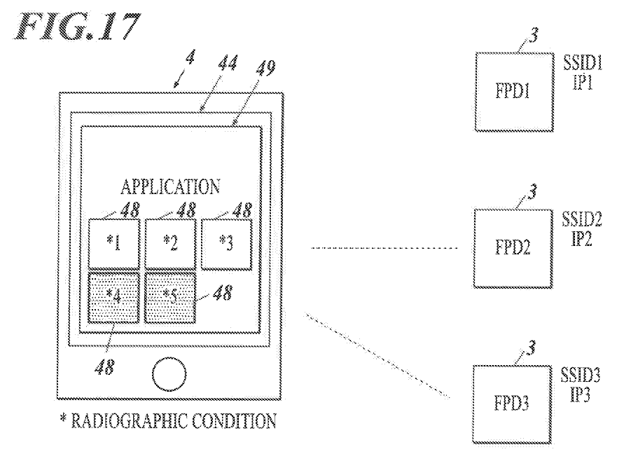

In a similar manner, the application 49 can determine the radiographic condition corresponding to the SSID detectable by the information processor from the associated information on the radiographic conditions and the communication identifiers as illustrated in the FIG. 16. As illustrated in FIG. 17, the selection button 48 of the radiographic condition corresponding to the SSID detectable by the information processor 4 is displayed in a mode different from that of the selection button 48 for the radiographic condition corresponding to the SSID undetectable by the information processor 4. This can inform the user of which radiographic condition is usable.

Hidden SSID and Limited Connection

If the SSID of the radiographic device 3 operating as the primary wireless device is visible from other wireless devices, this SSID is also visible from the wireless device owned by, for example, a patient. This is not desirable for security. In particular, smart phones and laptop computers have been popular in recent years. Since general patients can readily use these wireless communication devices, the visible SSID is not desirable.

The wireless devices can have the function that hides the SSID by the setting of the communicator 34 of the radiographic device 3 operating as the primary wireless device. Also in such a case, the application stores the SSID of the radiographic device 3 operating as the primary wireless device. Thus, the connection to the radiographic device 3 can be certainly established for transmission and reception of the data.

In the case that multiple applications are activated, the applications are controlled such that the communication with the radiographic device 3 specified by a subsequently activated application or the radiographic device 3 corresponding to the radiographic condition is established and the data is transmitted and received. In other words, a subsequent setting in the communicator 34 takes priority such that a subsequent connection setting can overwrite and the process for establishment of the communication is performed.

In the case that multiple applications are simultaneously executed, the communication with the radiographic device 3 specified by the active application or the radiographic device 3 corresponding to the radiographic condition is established for the transmission and reception of the data. In other words, after an application is specified and activated, the communication setting specified by the active application can overwrite and the process for establishment of the communication can be performed.

The combination of an ESSID and an access key can be used as a communication identifier.

Furthermore, as stated above, BSSID's may be checked and limited such that the communication with the BSSID other than the preregistered BSSID is not established. For example, a MAC address assigned to the communicator 34 of the radiographic device 3 can be used as a BSSID.

For example, a BSSID, an IP address, and a host name can be used as an address.

Although not illustrated, a setter of the information processor 4 or the console 2 connected with the information processor 4 can set the communication identifier and the address or associate the icon 46 or the selection button 48 of the application with the radiographic condition.

In this manner, the hardware processor 41 of the information processor 4 reads the corresponding combination information and establishes the communication based on the read combination information after activation of the application or operation of the icon 46 or the selection button 48.

After establishment of the communication, various data is transmitted and received to/from the connected radiographic device 3 in response to the stored address. Thus, the number of images taken and stored by the radiographic device 3 and the images taken can be checked on the screen of the information processor.

As described above, the communication system S according to the present embodiment can generate the data of the radiographic image in response to reception of radiation. The system S includes a radiographic device 3 operable as a primary device and an information processor 4 operable as a secondary device in wireless communication.

The information processor 4 includes:

a memory 43 that stores combination information of a communication identifier of the radiographic device 3 (for example, at least one of a BSSID, an ESSID, and an access key) and an address (for example, at least one of the BSSID, an IP address, and a host name);

an operation interface 45 that is associated with the combination information and is operated by a user; and

a communicator establishing communication with the radiographic device 3 based on the communication identifier included in the combination information corresponding to an operation on the operation interface and performing at least one of the transmission and reception of the data to and from the radiographic device 3 based on the address.

The wireless communication between the radiographic device and the information processor can be certainly established even if multiple radiographic devices are in use.

### Example 1

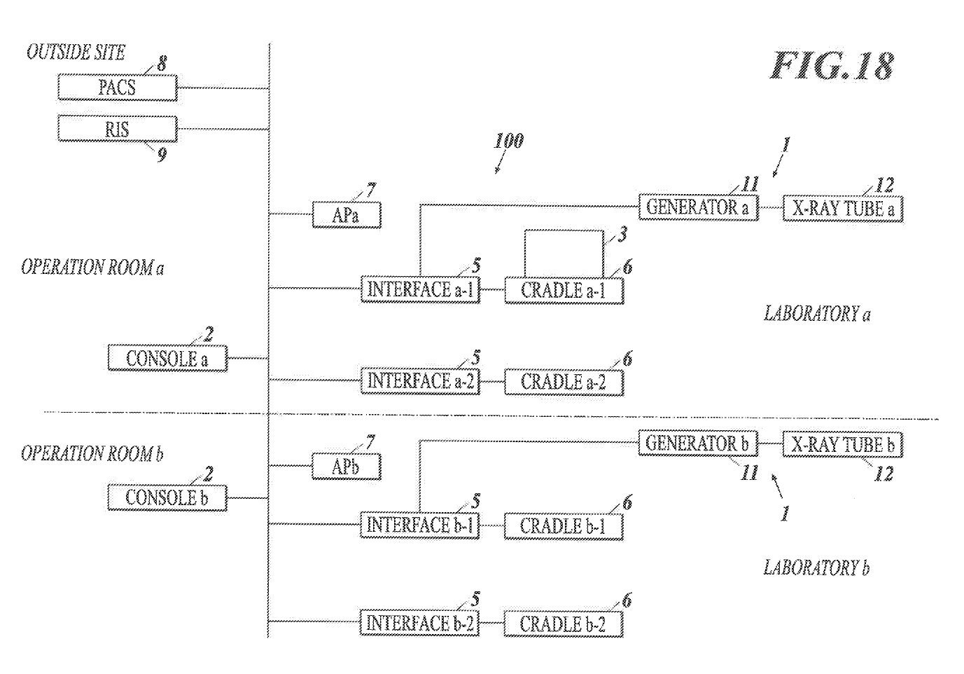

A more specific example of the radiographic system 100 according to the present embodiment will now be described. FIG. 18 is a block diagram of an operation of the radiographic system 100 in a hospital according to the present example. In FIG. 18, the information processor 4 is not depicted.

As illustrated in FIG. 18, the radiographic system 100 according to the present example is disposed in a room (laboratory “a”) in an establishment and includes the components according to the present embodiment and a pair of interfaces 5, a pair of cradles 6, and an access point (hereinafter “AP 7”).

The paired interfaces 5 are both connected to a console 2, and the paired cradles 6 are connected to the respective interfaces 5. Any of the two cradles 6 receives a radiographic device 3, which is then connected to, for example, the console 2.

An irradiator 1 is connected to, for example, the console 2 via one of the interfaces 5.

The AP 7 is connected to the console 2.

The console 2 according to the present example can detect a device connected to a network and display a network connection diagram as illustrated in FIG. 18 on the display of the console 2.

The console 2 can modify the names of the components and the IDs of the displayed devices to modify display for ready understanding of a user. For example, the cradles 6 disposed in the laboratory “a” can be displayed by reference numerals and symbols with suffixes like “cradle a-1”. Each component will be sometimes described below with the suffix as required.

In the present example, the irradiator 1, the console 2, the paired interfaces 5, the paired cradles 6, the AP 7 are also disposed in a different room (laboratory “b”). The radiographic device 3 can be moved from the laboratory “a” into the laboratory “b” and is inserted into the cradle 6 such that the radiographic system 100 works. The radiographic systems 100 in the respective laboratories are connected to a PACS 8 and a RIS 9 in an outside site.

In the case that the sites (laboratories) of the radiographic systems 100 needs to be distinguished from each other in the description, different reference numerals and symbols may be used, such as “console a” and “cradle a-1”.

### Example 1-1: Initial Setting

At the shipment of the radiographic device 3 from a plant, the radiographic device 3 is assigned radiographic device identifiers, such as BSSID and MAC address included in the communication identifier according to the present invention, specific to the device and default values for network (NW) setting (combination information according to the present invention, for example, the combination of an ESSID and an access key or an IP address). In use of the radiographic device 3 for radiography, the assigned default values for network setting should be modified according to the situation of the establishment used.

In use of the radiographic device 3 operable as both the primary wireless device and the secondary wireless device, the mode of use as primary wireless device or the secondary wireless device and the network setting for use as the primary wireless device or the secondary wireless device needs to be specified.

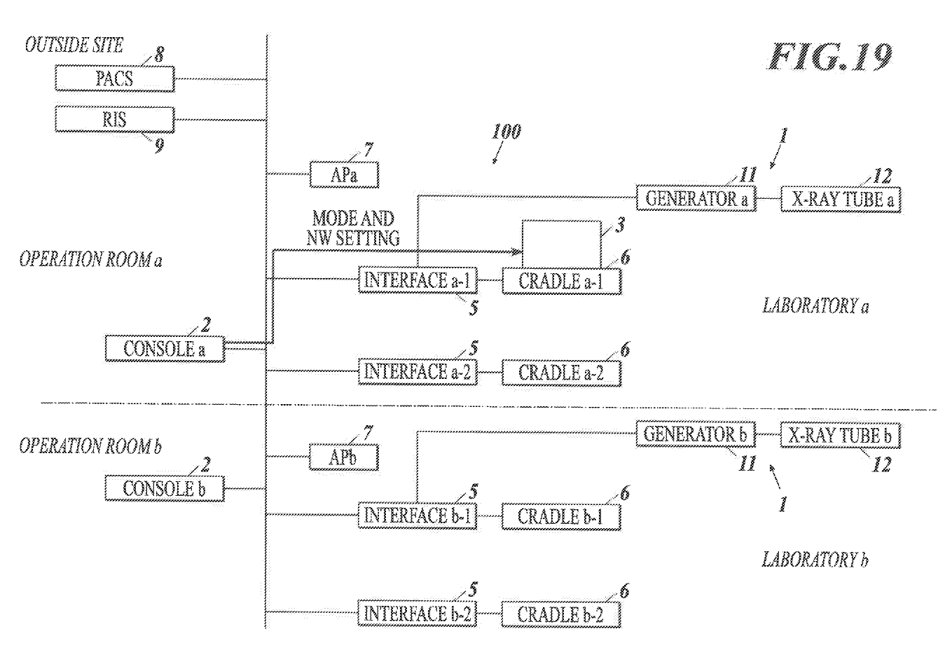

For example, when the radiographic device 3 is inserted in the cradle 6 and connected to the console 2 in the radiographic system 100 according to the present example as illustrated in FIG. 19, the mode of use of the radiographic device 3 as the primary wireless device or the secondary wireless device and the network setting of the radiographic device 3, for example, the SSID, access key, and IP address, are received from the console 2 and are stored in the console 2 and the radiographic device 3 in a non-volatile manner.

The radiographic system 100 according to the present example includes two cradles 6. For example, after a cradle a-1 receives the radiographic device 3, the radiographic device 3 can be defined as the primary wireless device. After the cradle a-2 receives the radiographic device 3, the radiographic device 3 can be defined as the secondary wireless device. Such a definition can be made on a setting software program installed in, for example, the console 2.

In this manner, the appropriate network setting for the radiographic device 3 allows the use of one radiographic device 3 (i) in the mode of the secondary wireless device, (ii) in the mode of the primary wireless device, and (iii) in combination with a different device in a different room or in combination with multiple visiting carts depending on the situations of the establishment used.

In other words, in use in the mode of the secondary wireless device, wireless devices can be mutually connected via the AP 7 in the laboratory. The radiographic device 3 set to operate in the mode of the secondary wireless device can be also connected to the network simultaneously via the AP, like other wireless devices.

In the case of use in the mode of the primary wireless device, one radiographic device 3 can be used in combination with multiple examination rooms or visiting carts. Thus, the radiographic device 3 can be efficiently operated.

Furthermore, the setting can be modified with less inconsistency with the intention of the user.

### Example 1-2: Modification of Setting of Radiographic Device

In the example of the radiographic system 100, after the radiographic device 3 is inserted into the cradle 6, a cradle identifier (device sub-ID) and a radiographic device identifier is broadcast in the network.

For example, after the radiographic device 3 is inserted into the cradle 6 and the connection to the cradle 6 is detected, the radiographic device 3 reads the cradle identifier written in the cradle 6 (device sub-ID) and may broadcast the cradle identifier and its own radiographic device identifier through the network.

Alternatively, after the cradle 6 receives the radiographic device 3 and the connection with the cradle 6 is detected, the cradle 6 reads the radiographic device identifier written in the radiographic device 3 and may broadcast the radiographic device identifier and its own cradle identifier (device sub-ID) through the network.

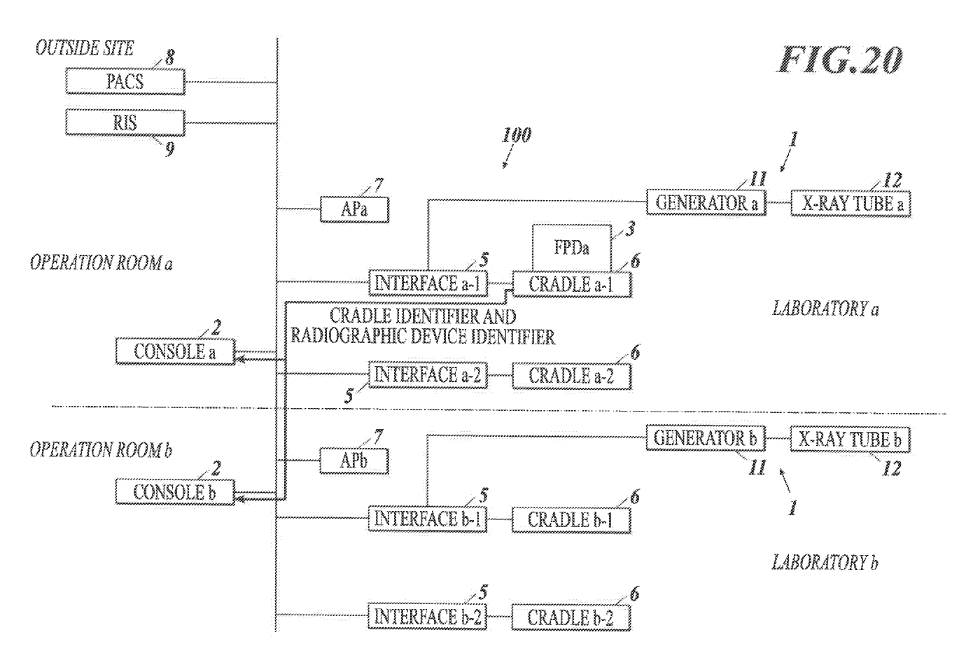

As illustrated in FIG. 20, after the cradle a-1 receives the radiographic device 3, the information on the cradle a-1 receiving the radiographic device 3 (for example, the cradle identifier of the cradle a-1) and the radiographic device identifier of the radiographic device 3 are broadcast through the network (transmitted to the consoles 2).

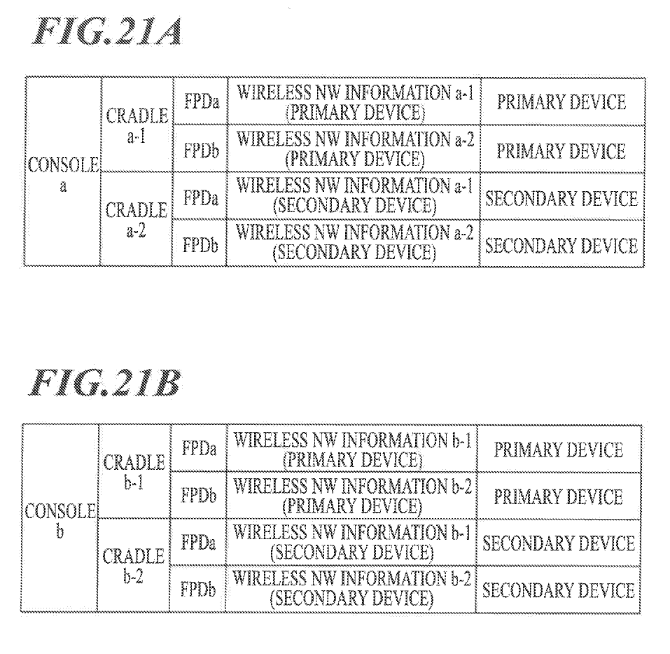

Each console 2 maintains the cradle identifiers of the cradles under the control by itself (for example, disposed in the same laboratory) as illustrated in FIG. 21A and FIG. 21B.

The console 2, which has received a cradle identifier under the control by itself in a request for setting, transmits the wireless network (NW) information to the radiographic device 3 for the connection of the radiographic device 3 to the console 2 in response to a request for setting.

The console 2 does not respond in the case that the console 2 receives a cradle identifier that is not under the control by itself.

The wireless network information transmitted in the case of the responded radiographic device 3 serving as the primary wireless device may be, for example, the ESSID and the access key of the primary wireless device or may be a list of the BSSIDs of the connectable secondary wireless devices.

The wireless network information transmitted in the case of the responded radiographic device 3 serving as the secondary wireless device may be, for example, the SSID and the access key of the connectable primary wireless device.

The network information in the case of the radiographic device 3 serving as the primary wireless device and the network information in the case of the radiographic device 3 serving as the secondary wireless device may include any other information. For example, the information necessary for certain connection through the network and the information to be checked for security can be appropriately added.

The wireless network information transmitted may include the mode defining the radiographic device 3 as the primary wireless device or the secondary wireless device.

After reception of a response to the request for setting from the console 2, the radiographic device 3 uses the wireless network information included in the response for its own setting.

For example, if the cradle a-1 in FIG. 20 receives a FPDa, the cradle identifier “cradle a-1” and the radiographic device identifier “FPDa” are broadcast through the network. The console “a” storing the information in FIG. 21A subsequently recognizes that the cradle a-1 controlled by the console “a” receives the FPDa from the identifier of the received FPDa and determines the FPDa to be used as the primary device.

The determination can be readily made by search of the information in FIG. 21A or FIG. 21B based on the cradle identifier and the radiographic device identifier.

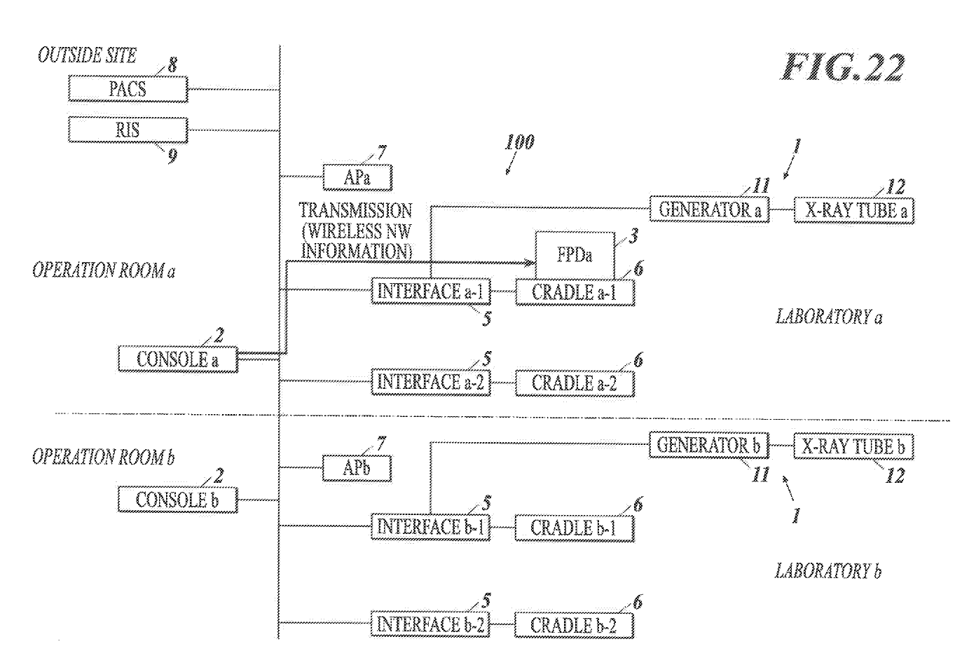

As illustrated in FIG. 22, the console “a” transmits the wireless network information functioning as the primary wireless device to the cradle a-1 or the radiographic device, i.e., FPDa.

The FPDa receiving the wireless network information evaluates the transmitted wireless network information as required and uses the wireless network information for its wireless network setting.

### Example 1-3: Definition as Secondary Device

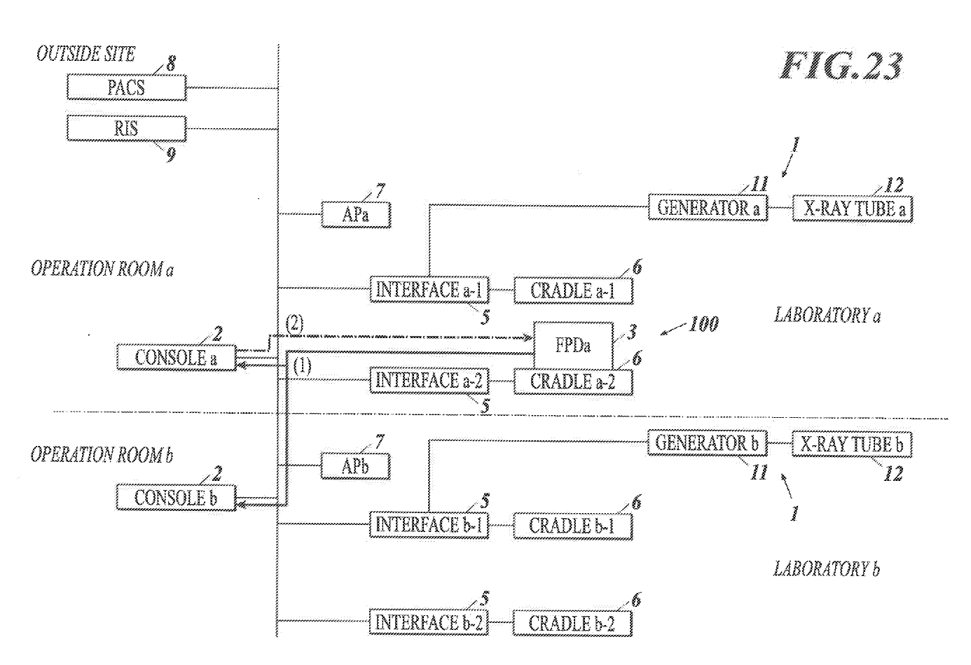

As illustrated in FIG. 23, the wireless network information in the radiographic system 100 according to the example may be determined such that the radiographic device 3 is used as the secondary wireless device if the cradle a-2 defined as the secondary wireless device receives the radiographic device 3.

In this manner, the radiographic device 3 can be certainly defined as the primary wireless device or the secondary wireless device. As desired by the user, the radiographic device 3 can be used as the primary wireless device or the secondary wireless device.

In other words, in the case that the cradle a-2 in FIG. 23 receives the FPDa, the cradle identifier “cradle a-1” and the radiographic device identifier “FPDa” are broadcast through the network. The console “a” storing the information in FIG. 21A subsequently recognizes that the cradle a-1 under the control by itself receives the FPDa and determines the FPDa to be used as the secondary device from the identifier of the received FPDa.

The determination can be readily made by search of the information in FIG. 21A or 21B based on the cradle identifier and the radiographic device identifier.

The console “a” transmits the wireless network information functioning as the secondary wireless device to the cradle a-2 or the FPDa inserted into the cradle a-2 as in FIG. 22, although not illustrated in FIG. 23.

The radiographic device or FPDa receiving the wireless network information evaluates the transmitted wireless network information as required and uses the wireless network information for its own wireless network setting.

In this manner, the radiographic device 3 can be certainly defined as the primary wireless device or the secondary wireless device. The user can use the radiographic device 3 as the primary wireless device or the secondary wireless device.

The radiographic devices 3 can be inserted into different cradles and defined as the primary wireless devices or the secondary wireless devices. For example, the radiographic device 3 defined as the primary wireless device is inserted into the cradle a-1, or the radiographic device 3 defined as the secondary wireless device is received by the cradle a-2. Thus the setting can be modified with less inconsistency with the intention of the user.

### Example 1-4: Use in Combination with Different Console

In the case that the radiographic system 100 according to the example is used in a different room (laboratory “b”), the settings according to the examples 1-1 to 1-3 may also be made on the console 2 and the cradle 6 in the other room.

Specifically, the same software program as that of the console “a” is installed in the console “b”. If a cradle b-1 receives the radiographic device 3, the radiographic device 3 is defined as the primary wireless device. If a cradle b-2 receives the radiographic device 3, the radiographic device 3 is defined as the secondary wireless device.

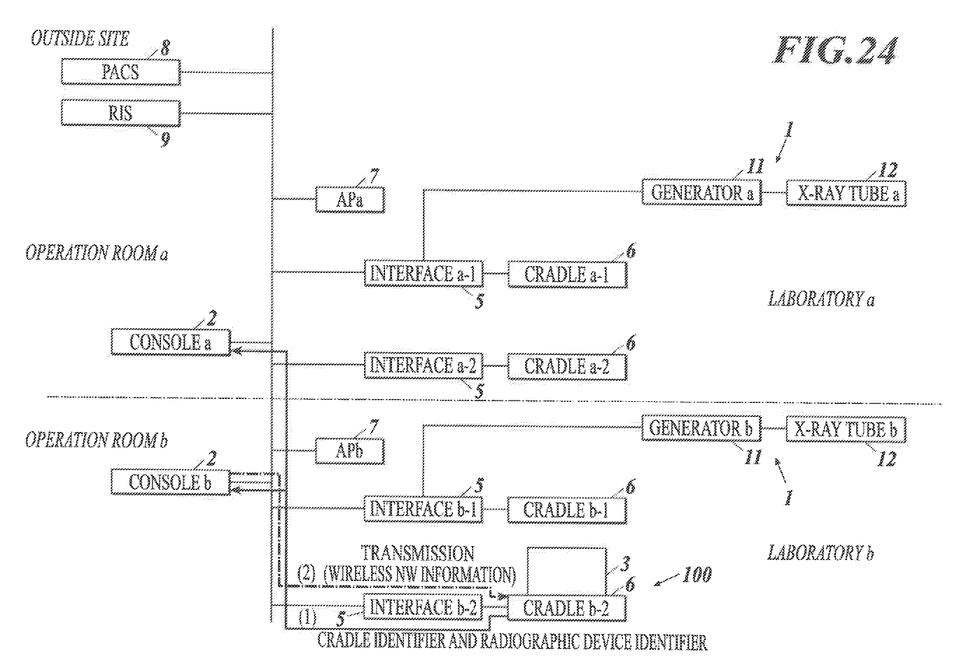

As illustrated in FIG. 24, (i) after the radiographic device 3 is inserted into the cradle 6, the radiographic device 3 reads the cradle identifier written in the cradle 6 and broadcasts the cradle identifier and the radiographic device identifier through the network, and (ii) in the case that the console “b” receives the cradle identifier under the control by itself, the console “b” responds to the request for setting from the radiographic device 3.

In this manner, the radiographic device 3 in the other laboratory or under the control by the other console 2 can be certainly defined as the primary wireless device or the secondary wireless device in the same procedure. As desired by the user, the radiographic device 3 can be used as the primary wireless device or the secondary wireless device.

The radiographic devices 3 are inserted into different cradles and defined as the primary wireless devices or the secondary wireless devices. For example, the radiographic device 3 defined as the primary wireless device is inserted into the cradle b-1, and the radiographic device 3 defined as the secondary wireless device is inserted into the cradle b-2. Thus the setting that is not intended by the user can be certainly reduced.

Besides the use in a different room (laboratory “b”), for example, in the use in a different visiting cart, the radiographic device 3 performs the same setting and procedure with the console 2 disposed in the visiting cart. Thus, as desired by the user, the radiographic device 3 can be used as the primary wireless device or the secondary wireless device.

### Example 1-5: Limited Combination with Console Defined as Primary Wireless Device

If the radiographic system 100 according to the example includes multiple radiographic devices 3 defined as primary wireless devices, radiowave interference may be generated, resulting in unstable communication. In order to reduce such radiowave interference, the number of radiographic devices 3 defined as primary wireless devices and connectable to one console 2 may be limited to one.

To solve such a problem, in the case that the radiographic device 3 defined as the primary wireless device is inserted into the cradle 6 and receives the wireless network information including an SSID different from that stored in the radiographic device 3 in a non-volatile manner from the console 2 in the example 1-1, the wireless network information need not be reflected (or not be overwritten).

In the illustrated layout in FIG. 18, the console 1 in the laboratory “a” stores the cradle identifiers in FIG. 21A, while the console 2 in the laboratory “b” stores the cradle identifiers in FIG. 21B. The tables in FIGS. 21A and 21B indicate that the wireless network information a-1 (of the primary device) is assigned to the radiographic device 3 and the radiographic device 3 is used as the primary wireless device, for example, if the radiographic device 3 is inserted into the cradle a-1 under the control by the console “a”.

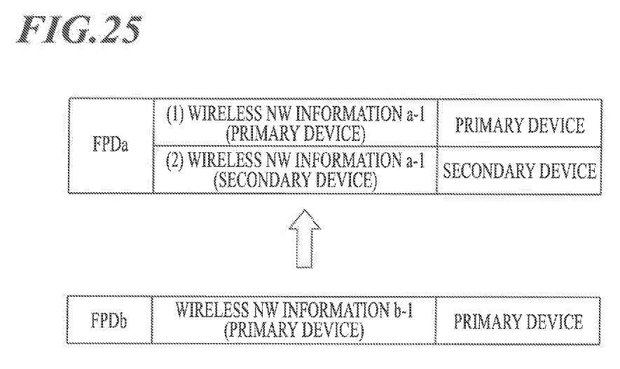

In a specific situation, the network settings in the upper table of FIG. 25 can be stored in the memory 35 of the radiographic device 3.

In these settings, not only the actual network settings but also the network settings of the radiographic devices serving as the primary wireless devices or the secondary wireless devices are stored, such as (i) the wireless network information in the case of the radiographic device serving as the primary wireless device and (ii) wireless network information in the case of the radiographic device serving as the secondary wireless device in the upper table of the FIG. 25.

Whether or not to permit the definition as a different primary wireless device in the wireless network information may be specified and stored in the memory.

“In a specific situation, the network settings in the upper table of FIG. 25 are stored in the memory 35 of the radiographic device 3” refers to the settings in, for example, a mode different from that of an operation by a general user and a mode that is used by a person other than the user at the installation of or the modification of the settings of the radiographic system 100 (for example, the maintenance person from a manufacturer) and cannot be used by the general user in a typical operation. Such modes may be provided as part of the functions of the setting software program described above.

In the case that the setting that does not permit the definition as a different primary wireless device in the wireless network information are specified and the wireless network information other than that of the primary wireless device stored in the radiographic device 3 is received by the radiographic device 3 in the procedure according to the example above, the wireless network setting of the radiographic device 3 is not modified.

As illustrated in FIGS. 21A, 21B, and 25, the FPDa defined as primary wireless device in the console “a” and not permitted to be defined as a different primary wireless device in the wireless network information is not assigned the network setting for operating in cooperation with the console 2 after the insertion into the cradle b-1 under the control of the console “b”.

In other words, as illustrated in the upper table of FIG. 25, the memory 35 of the FPDa pre-stores the network setting (wireless NW information a-1 (primary device)) for the primary wireless device operating in cooperation with the console “a”. After the FPDa is inserted into the cradle b-1 configured to define the FPDa as the primary wireless device operating in cooperation with the console “b”, the procedure described above is performed and the FPDa receives the response that the FPDa is assigned the network setting for the primary wireless device (wireless NW information b-1 (primary device)) from the console “b”.

Reversely, the FPDa storing a network setting for the primary device different from that in the console “b” and not permitted to be defined as a different primary wireless device in the wireless network information in the console “b” is not assigned the network setting for the primary wireless device.

Even if the definition of the radiographic device 3 as a different primary wireless device in the wireless network information is not permitted, the network setting for the primary wireless device stored in the radiographic device 3 defined as the secondary wireless device operating in cooperation with the console “a” or the console “b” may match the network setting in the console “a” or console “b”. This radiographic device 3 can operate as the primary wireless device in cooperation with the console “a” after insertion into the cradle a-1 under the control by the console “a”.

In this manner, the devices operating as the primary wireless devices can be limited, and radiowave interference resulting in unstable communication can be certainly reduced.

In particular, the devices other than the radiographic device is often used in a fixed environment in view of the difficulties with use in different installation environments due to the utility issues, such as size, weight, required power supply, and water.

In contrast, the radiographic device 3 is movable for use. For example, multiple radiographic devices 3 may be used in a single laboratory for the radiography generating a long image. If every radiographic device 3 is defined as the primary wireless device regardless of environments, a large number of primary wireless devices beyond expectation may concentrate in the laboratory.

In the case that the primary wireless devices concentrate in the laboratory, radiowave interference may be caused, resulting in a communication failure. In the present example, the combination of the radiographic device 3 defined as the primary wireless device with the information processor 4 is limited, and the communication failure due to the radiowave interference can be certainly reduced.

### Example 1-6: Setting Confirmation

In the radiographic system 100 according to the example above, the user cannot sometimes determine whether the network setting of the radiographic device 3 as the primary wireless device or the secondary wireless device is certainly performed as intended by an administrative operator.

An informer may be thus provided that notifies of the radiographic device 3 inserted into the cradle a-1 defined as the primary wireless device on the successful completion of the network setting of the radiographic device 3 as the primary wireless device or the secondary wireless device as intended by the administrative operator or on the unsuccessful setting of the radiographic device.

Specifically, if the setting of the radiographic device 3 is completed, the radiographic device 3 or the cradle 6 notifies the user of the completion.

The notice of completion can be made by, for example, light, sound, or vibration.

If the setting of the radiographic device 3 is not successfully completed, the radiographic device 3 or the cradle 6 notifies the user of an error.

The notice of error can be made by, for example, light, sound, or vibration different from that in the notice of completion.

In the case of the unsuccessful setting of the radiographic device 3, the notice of setting completion need not be made but the notice of error may be merely suspended.

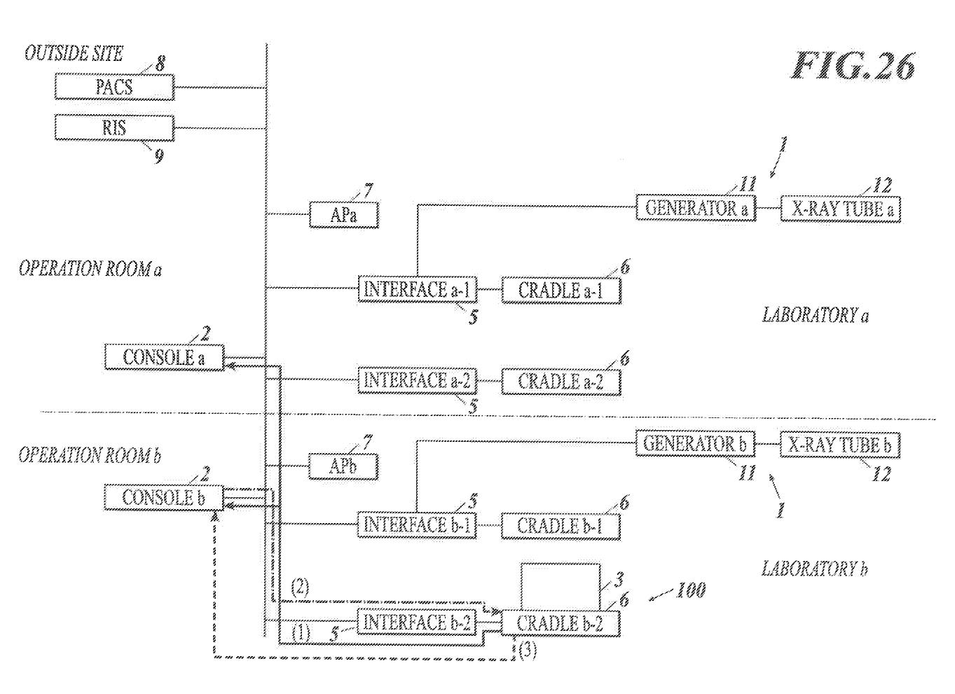

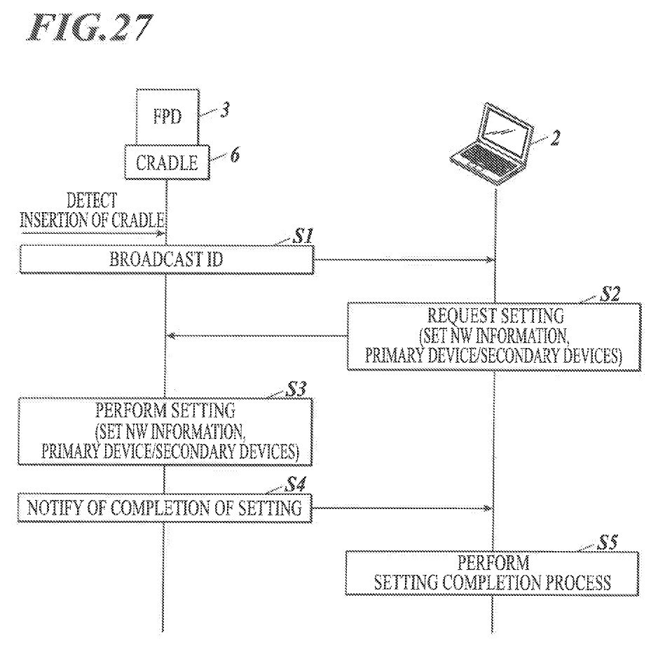

Instead of the radiographic device 3 or the cradle 6, the console 2 may give notice. As illustrated in FIGS. 26 and 27, the console 2 makes the notice of completion as follows.

(i) After detection of the radiographic device 3 inserted into the cradle 6, the cradle identifier and the radiographic device identifier are broadcast (Step S1).

(ii) The console 2 responds to the request for setting (Step S2), and then the radiographic device 3 is set (Step S3).

(iii) Based on the completed setting, the radiographic device 3 or the cradle 6 transmits a signal indicating that the setting is completed to the console 2 (Step S4). In response to the completion signal, the console 2 performs the process for completion of setting (Step S5).

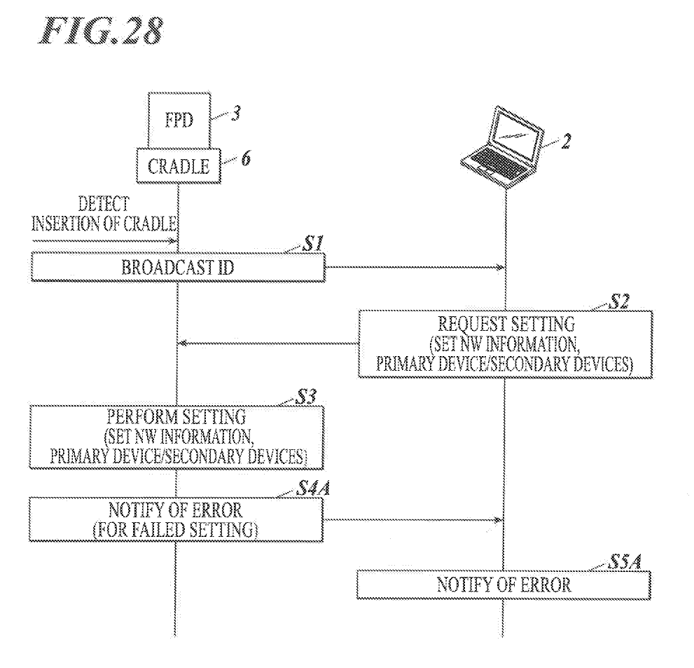

As illustrated in FIG. 28, the console 2 gives notice of error as follows: the cradle identifier and the radiographic device identifier are broadcast (Step S1). The console 2 then responds to the request for setting (Step S2) followed by the trial of the setting of the radiographic device 3 (Step S3). Based on the unsuccessful setting of the radiographic device 3, the radiographic device 3 or the cradle 6 transmits an error signal indicating that the setting cannot be completed to the console 2 (Step S4A). In response to the error signal, the console 2 gives the notice of error (Step S5A).

For example, if the radiographic device 3 inoperable as the primary wireless device is inserted into the cradle a-1 defined as the primary wireless device, the user is informed of the unsuccessful setting of the primary wireless device by lack of sound. Alternatively, the user is informed of the unsuccessful setting of the primary wireless device by a sound different from that in the notice of successful setting of the primary wireless device.

The radiographic device 3 inoperable as the primary wireless device is, for example, the radiographic device 3 rejected in “EXAMPLE 1-5: LIMITED COMBINATION WITH CONSOLE DEFINED AS PRIMARY WIRELESS DEVICE” or the radiographic device 3 including only the communicator for the secondary wireless device but not for the primary wireless device.

The sound for the notice of error may be modified for each reason for unsuccessful setting of the primary wireless device such that the user is informed of the reason for unsuccessful setting of the radiographic device as the primary wireless device.

For example, the successful setting of the radiographic device as the primary wireless device may be noticed by a voice message “the radiographic device 3 has been defined as the primary wireless device”. The unsuccessful setting of the primary wireless device may be noticed by a voice message “the radiographic device 3 is inoperable as the primary wireless device and cannot be defined as the primary wireless device”.

The successful or unsuccessful setting of the radiographic device may be noticed on the display of the console 2.

In this manner, the user can certainly know whether the radiographic device can be defined as the primary wireless device.

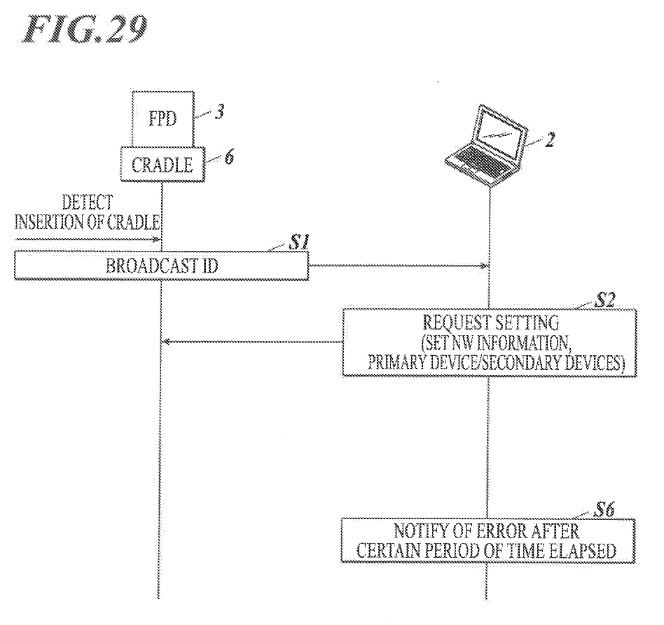

As illustrated in FIG. 29, the console 2 may have a function to perform Step S6, i.e., to notice the unsuccessful setting in the case that the notice of completion is not given during a predetermined period of time after the request for setting.

The notice of the unsuccessful setting can be given by, for example, light, sound, or vibration.

In this manner, in the case that the successful completion of the setting cannot be confirmed due to a communication failure, the administrative operator can be certainly informed of the unsuccessful setting, and the radiographic device is prevented from using contrary to the intention of the administrative operator.

In the example 1-5, an informer may be provided that notices the successful or unsuccessful setting of the radiographic device 3 as the primary wireless device after the insertion of the radiographic device 3 into the cradle defined as the primary wireless device.

Specifically, the radiographic device 3 or the cradle 6 notifies of the completion after the successful setting of the radiographic device 3.

The notice of completion can be given by, for example, light, sound, or vibration.

In the case of unsuccessful setting of the radiographic device 3, the radiographic device 3 or the cradle 6 notifies the user of an error.

The notice of error can be given by, for example, the light, sound, or vibration different from that in the notice of completion.

In the case of the unsuccessful setting of the radiographic device 3, the notice of error need not be given but the notice of setting completion may be merely suspended.

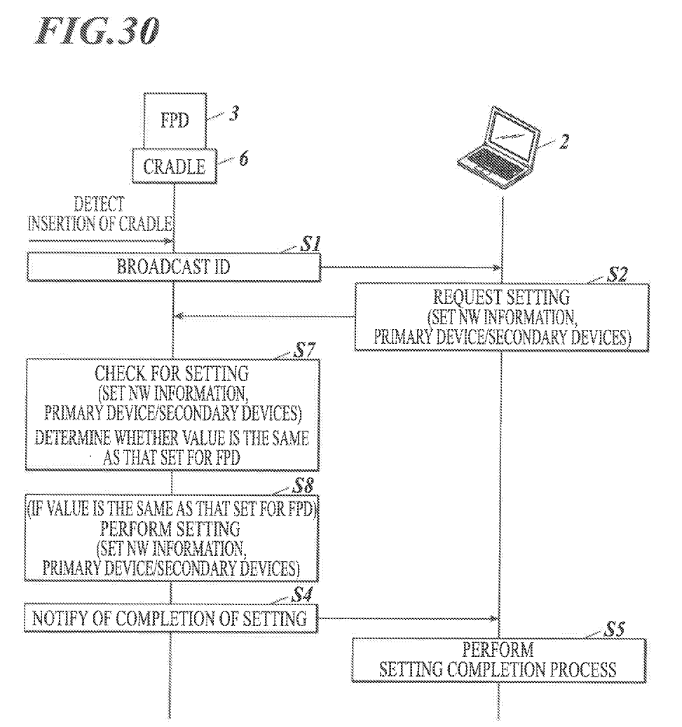

The console 2 may give notice instead of the radiographic device 3 or the cradle 6. As illustrated in FIG. 30, the console 2 gives the notice of completion as follows: the cradle identifier and the radiographic device identifier are broadcast (Step S1). The console 2 responds to the request for setting (Step S2). Whether the network information and the mode received from the console is the same as those defined in the radiographic device 3 is determined (Step S7). If the network information and the mode is determined to be the same, the setting of the radiographic device 3 is performed (Step S8). Based on the completed setting, the radiographic device 3 or the cradle 6 transmits a signal indicating that the setting is completed to the console 2 (Step S4). In response to the completion signal, the console 2 performs the process for completion of the setting (Step S5).

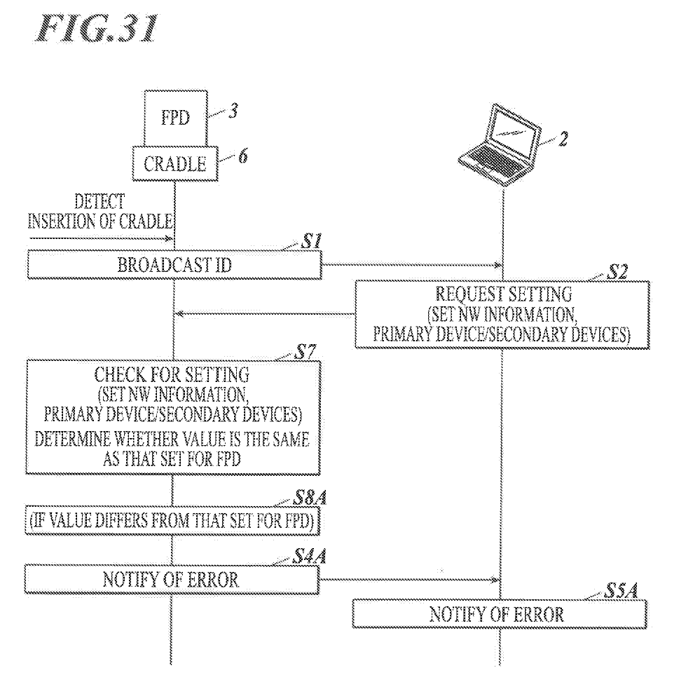

As illustrated in FIG. 31, the console 2 notifies of an error as follows: the cradle identifier and the radiographic device identifier are broadcast (Step S1). The console 2 responds to the request for setting (Step S2). Whether the network information and the mode received from the console are the same as those defined in the radiographic device 3 is determined (Step S7). If the network information and the mode are determined to be different, the setting of the radiographic device is not performed (S8A). Based on the unsuccessful setting of the radiographic device 3, the radiographic device 3 or the cradle 6 transmits an error signal indicating that the setting cannot be completed to the console 2 (Step S4A). In response to the error signal, the console 2 performs an error process (Step S5A).

In this manner, in the case that the radiographic device cannot be defined the primary wireless device as specified, the user can be certainly informed of the unsuccessful setting, and the radiographic device can be prevented from using contrary to the intention of the user.

If the radiographic device cannot be defined as the specified primary wireless device in the procedure described in the example 1-5, the notice of error different from that in the example may be given. Specifically, the light, sound, or vibration different from that in the notice of error according to the example is used.

Alternatively, the reason for unsuccessful setting may be noticed by a voice message “this radiographic device 3 cannot be used as the primary wireless device in combination with this information processor 4”.

In this manner, in the case that the radiographic device cannot be defined as the primary wireless device as specified, the different type of notice of error can be used to inform the user of the reason for unsuccessful setting.

### Example 1-7: Acquisition of Radio Field Intensity

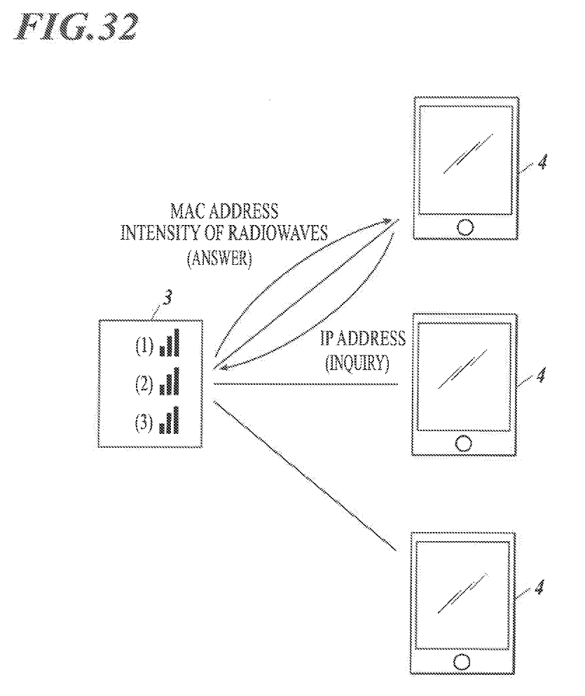

The radiographic device 3 defined as a single primary wireless device is connected to multiple information processors 4, the radio field intensities of the information processors 4 should be displayed on the information processors 4 as illustrated in FIG. 32. However, the information processor 4 activating an application cannot display the radio field intensity on the display in some cases. The application cannot even acquire the radio field intensity from such an information processor 4 in some cases.

The radiographic device 3 connected to the information processors 4 can acquire the radio field intensities of the information processors 4. Thus, the application can preferably acquire the radio field intensities of the information processors 4 from the radiographic device 3.

The application usually includes four layers, namely an electric signal layer L1, a wireless layer L2, a communication layer L3, and an application layer L4. When the radio field intensity is acquired from the radiographic device 3 through the operation of the application, the application layer L4 transmits the IP address to the radiographic device 3. The radiographic device 3 is defined as the primary wireless device and must select the radio field intensity of a targeted information processor from the radio field intensities contained in the information. However, the protocol for transmission of the radio field intensity to the application is defined only by the wireless layer L2 and corresponds only to the MAC address. In other words, the application installed in the information processor 4 can acquire the radio field intensity from the radiographic device 3 but cannot display the radio field intensity in some cases.

To solve such a problem, the targeted radio field intensity may be identified and transmitted in reference to an ARP table representing the correspondence between the MAC address and the IP address.

In this manner, the information processor 4 including the application inoperable to directly acquire and display the radio field intensity of the information processor 4 can display the radio field intensity.

The communication layer L3 is vulnerable, and the ARP table may be temporarily invisible. Thus the ARP may be cached.

In reference to radio field intensities, the connection to or the operational input from the information processor 4 having a good radio field intensity may be preferentially accepted.

### Example 1-8: Display of Disconnected Communication

If the console 2 is disconnected from the radiographic device 3 operating as the primary wireless device, the user who is not informed of the disconnection may continue his or her task.

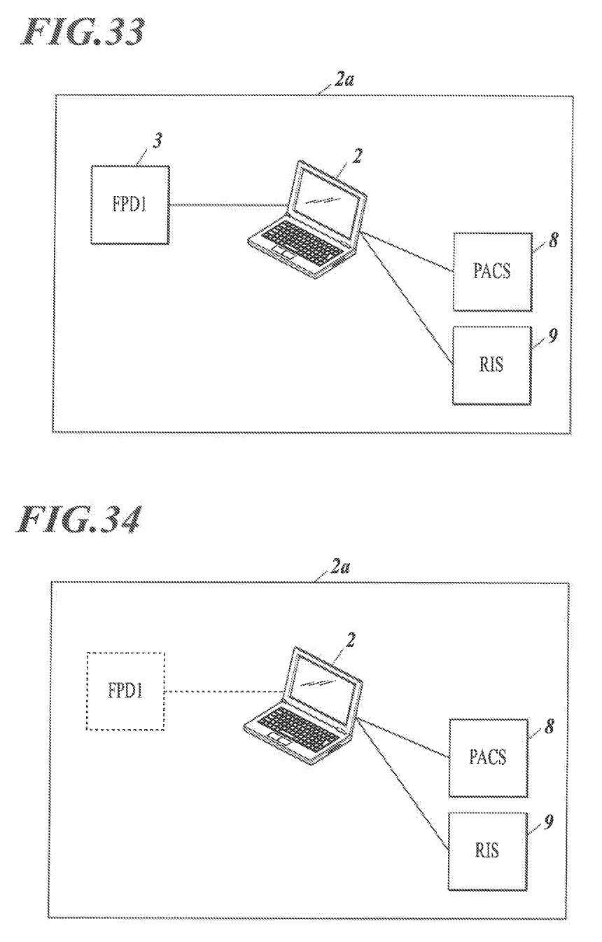

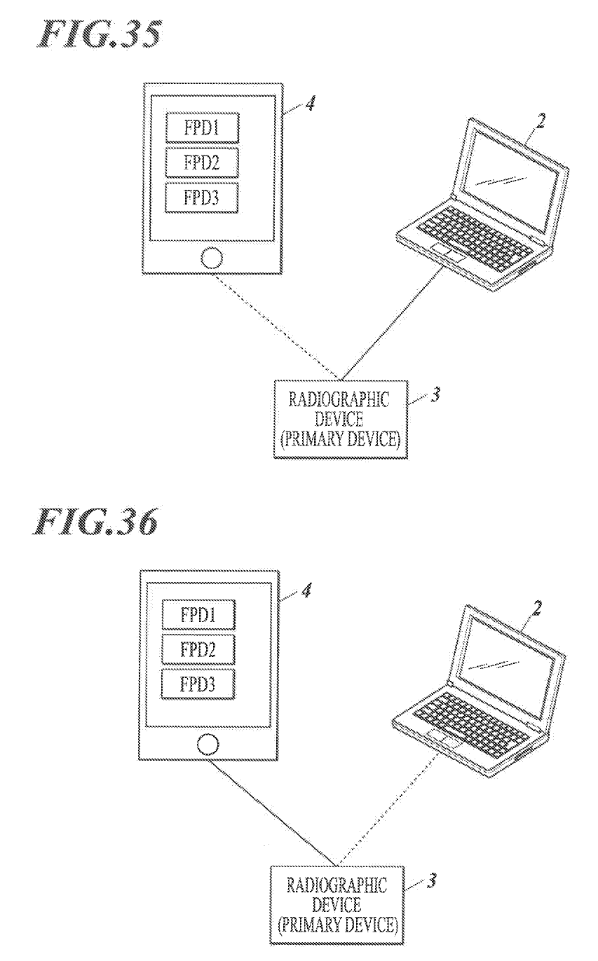

To solve such a problem, the console 2 and the information processor 4 may have a function that monitors the state of the connection at predetermined time intervals. The console 2 and the information processor 4 may also have a function that varies the display modes of the state of connection in the network connection diagram on the display 2a from the mode indicating the connection of the console 2 with the radiographic device 3 in FIG. 33 to the mode indicating the connection of the console 2 with the invisible radiographic device 3 in FIG. 34 in the case of the undetected connection with the primary wireless device.

In this manner, the user is prevented from continuing a task without knowing that the console 2 is disconnected from the primary wireless device.

In the example 1-8, the radiographic device 3 need not disappear from the display of the console 2. Alternatively, the radiographic device 3 may be indicated, for example, by dotted lines, which indicate unestablished connection, as illustrated in FIG. 34.

After the connection is not established during a predetermined period, the radiographic device 3 may disappear from the display of the console 2.

In an alternative configuration, the user may check for the connection on the console 2 and instruct the radiographic device 3 to disappear.

The radiographic device 3 that disappears from the display after disconnection may preclude the confirmation on the originally desired connection. In this example, the desired connection that is lost is indicated by dotted lines, which indicate unestablished connection. Thus, the administrative operator can check for the connection and perform the operation for recovery of the connection, resulting in a reduction in the downtime.

In the example 1-8, the radiographic device 3 may still appear on the console 2 or the display mode may be still maintained immediately after the connection is lost. In other words, the radiographic device 3 may disappear from the console 2 or the display mode may be switched after a predetermined time from the loss of connection elapses.

The connection in wireless communication may be lost instantaneously or for a few seconds and may be recovered by automatic reconnection. The loss of connection in such a case is not serious. The invisible radiographic device or the switching of the display mode in this case causes the user to misunderstand that the connection is lost. The setting described above can reduce the misunderstanding of the user.

### Example 1-9: Switching of Connection to Information Processor

The radiographic device 3 as the primary wireless device, which operates as the primary wireless device, can establish the communication with multiple secondary wireless devices. Thus, multiple controllers capable of controlling the radiographic device 3, such as the console 2 and the information processor 4, may simultaneously establishes the communication with the radiographic device 3.

Such simultaneous establishment of the communication with and control by the controllers may enhance usability because the radiographic device 3 can be operated by users in multiple sites. However, different operators in these sites may instruct the radiographic device 3 to perform operations inconsistent with their intentions, resulting in unintended radiographic capturing.

Thus, the control should be permitted to a selected one of the controllers in established communication with the radiographic device 3 used as the primary wireless device.

The controller can be selected by the software program installed in the radiographic device 3 to manage controllers and switch the controller authorized to control the radiographic device 3 separately from the management of the device with which the communication is established.

Alternatively, the controller may be selected by switching of connection ports assigned to target communication devices using software.

The selection can be performed by operating a button on the radiographic device 3 or by setting the data on the console 2 or the information processor 4.

The radiographic device 3 used in a particular operation mode may select a particular controller for automatic connection with the controller in the manner described above.

In the case that the radiographic device 3 cannot transmit and receive the data to/from the controller selected for automatic connection, the radiographic device 3 may be connected to another controller. Such a case may incorporate a process that informs the administrative operator of the unsuccessful transmission and transmission of the data and asks the administrative operator whether the connection to the other controller should be selected, before the connection of the radiographic device 3 to another controller. The completion of the connection may be transmitted to the other controller.

The radiographic device 3 according to the present embodiment may have a function that automatically detects radiation without communication with, for example, the console 2 to generate image data and store the image data in the memory before the image data can be transmitted (a radiographic device memory image capturing mode).

The radiographic device memory image capturing mode can be selected by operating the button on the radiographic device 3 or by setting the data on the console 2 or the information processor 4.

The radiographic device 3 uses a connection port “a” for connection to the console 2 and a connection port “b” for connection to the information processor 4. Different connection ports may be provided for other connecting devices.

In the case that the radiographic device memory image capturing mode is selected, the radiographic device 3 can be automatically switched to a connection port preset on the software program of the radiographic device 3.

For example, the radiographic device 3 in the radiographic device memory image capturing mode can be automatically connected to the information processor 4 by an additional program incorporated in the software of the radiographic device 3 to switch the connection port to the connection port “b” for connection with the information processor 4.

The software program of the radiographic device 3 may include an additional program that automatically re-switches the connection port to the connection port “a” for connection to the console 2 during the OFF state of the information processor 4 or in the case of a problem in the communication with the information processor 4. In order to check for a failure in the communication with the information processor 4, an acknowledge request command (for example, a ping command) can be used on the software program of the radiographic device 3 to confirm the acknowledgement from the information processor 4.

For communication with the console 2 or the information processor 4 via, for example, a HTTP server for transmission and reception of the data, the radiographic device 3 used as the primary wireless device may access the server.

In this manner, the controller to be permitted to transmit and receive data is selected from the controllers with which the communication is established. The unintended operation on an unauthorized device can be thereby reduced.

The automatic selection of the controller to be permitted to transmit and receive the data can reduce the operation of the user and enhance the usability.

### Example 1-10: Mode of Power on or Power Off

The power-on and the subsequent power-off may switch, for example, the operational mode, which is defined before the power-off, as the primary wireless device or the secondary wireless device. The switched mode may be inconsistent with the intention of the user.

To solve such a problem, the initial setting of the mode may be stored in, for example, a non-volatile memory and may be recovered after the power-on and the subsequent power-off.

In other words, the radiographic device defined as the primary wireless device (or the secondary wireless device) before power-off operates as the primary wireless device (or the secondary wireless device) after the power-on.

In this manner, the connection before the power-off is maintained. Thus, the radiographic operation can be performed in the setting consistent with the intention of the user.

### Example 1-11: Operation in Loose Cooperation

For cooperation of the irradiator 1 (or the generator 11) with the radiographic device 3 generating image data in radiography, the irradiator 1 and the radiographic device 3 should be communicative with each other via wired or wireless connection and transmit and receive control signals for mutual control of the operations. However, the establishment of the connection is troublesome.

For example, in the field of the emergency medical services, immediate radiographic capturing is desired depending on the condition of a patient. The sites where the images of patients are taken differ from each other depending on the conditions of the patients. Thus the portability of the devices and the quick radiographic capturing after the arrival of the patient are desired.

In other words, the radiography is desired that uses only the radiographic device 3 of a panel type, the portable information processor 4 checking for the status of the radiographic device 3, and the movable irradiator without establishment of communication for close cooperation of the devices and without any procedure before the capturing of an image starts.

In view of such a challenge, the radiographic device 3 may have a function that captures an image in a non-cooperative mode automatically starting to take an image after the detection of radiation without reception of control signals from an external device, such as the console 2.

In this manner, an image can be taken without establishment of the communication and the procedures before the image is taken. Thus, the time required before capturing of an image can be considerably shortened.

Of course, if the communication can be immediately established, an image may be taken in a cooperative mode where the irradiator 1 cooperates with the console 2 that permits the irradiation and determines the timing of irradiation.

### Example 2-1: Prevention of ID Overlap in Setting

If the radiographic device 3 defined as one primary wireless device is assigned the same SSID as that assigned to a communication device defined as another primary wireless device, the devices cannot be properly identified and the user may connect the device to an unintended communication target.

To solve such a problem, predetermined characters may be added to the tail of the ESSID. In this case, an initial setter assigns different ESSIDs to the primary wireless device and the secondary wireless device.

The predetermined characters to be added are, for example, “PAP”.

In this manner, the SSIDs do not overlap. Thus the risk of connection to an unintended device can be reduced.

The radiographic devices 3 used as the primary wireless devices are assigned the ESSIDs with standardized strings. This facilitates the management by the network setter.

In the present example, the initial setter need not assign different ESSIDs to the radiographic devices 3 used as the primary wireless devices or the secondary wireless devices.

For example, the initial setter inserts the radiographic device 3 into the cradle a-1 in the laboratory “a” for definition of the radiographic device 3 as the secondary wireless device and adds the strings “room 1” to the ESSIDs of the console 2 and the radiographic device 3 to operate as the secondary wireless device in cooperation with the console “a” in the laboratory “a”.

In use thereafter, the radiographic device 3 is inserted into the cradle a-1 in the laboratory “a”. The radiographic device 3 can be then used as the secondary wireless device operating in cooperation with the console 2 in the laboratory “a”.

A subsequent procedure may be provided that confirms whether the radiographic device 3 is defined also as the primary wireless device. For example, a question “Is the radiographic device 3 subsequently used as the primary wireless device?” may be displayed on the console 2, and “yes” or “no” may be selected. If the radiographic device 3 is also defined as the primary wireless device, the network setting for the secondary wireless device “room 1” is added to the end of the ESSID. Then the additional strings for the primary wireless device is added to the ESSID like “room 1_PAP”.

The initial setter subsequently inserts the radiographic device 3 into the cradle a-1 in the laboratory “a” for definition of the radiographic device 3 as the primary wireless device and adds additional strings like “room 1_AP” to the ESSIDs of the console 2 laboratory “a” and the radiographic device 3 to operate as the primary wireless device in cooperation with the console 2.

In use thereafter, the radiographic device 3 is inserted into the cradle a-1 in the laboratory “a”. The radiographic device 3 can be then used as the primary wireless device operating in cooperation with the console 2 in the laboratory “a”.

In this manner, the setter can use the ESSID for the secondary wireless device to set the ESSID for the primary wireless device. Thus the setting procedure can be facilitated.

The ESSID assigned to the AP 7 for connection of the radiographic device 3 in the establishment is reused and assigned to the radiographic device 3 to operate as the primary wireless device. Thus the strings of the ESSID are standardized and the management by the network setter is facilitated.

In the present example, the cradle a-2 may be removed and the radiographic device 3 may be used only as the primary wireless device and not as the secondary wireless device operating in cooperation with the console 2. After this radiographic device 3 is re-inserted into the cradle a-1 used for setting of the secondary wireless device, the setting for the primary wireless device is overwritten in the radiographic device 3 and the console 2.

For example, the radiographic device 3 disposed on a visiting cart and operating as the primary wireless device in cooperation with the console 2 does not operate as the secondary wireless device in cooperation with the console 2 in some cases. In such a case, only one cradle 6 in cooperation with the console 2 can define the radiographic device 3 as the primary wireless device operating in cooperation with the console 2 according to the setting scheme described above.

A procedure may be provided to ask the cradle receiving the radiographic device 3 whether the radiographic device 3 may be defined as the primary wireless device before the setting is overwritten.

In this manner, the radiographic device 3 can be defined only as the primary wireless device by only one cradle. Thus, the cradles that are usually not used can be removed.

### Example 2-2: Check for Overlap of ID at Setting

If the radiographic device 3 defined as the primary wireless device is assigned the same SSID as that assigned to the radiographic device 3 defined as another primary wireless device, the connected device cannot be identified and the user may connect the device to an unintended communication target, as described in the example 2-1. To solve such a problem, whether the SSID of interest is used in the current communication network may be checked before the SSID is assigned to the radiographic device 3.

A procedure may be provided to check whether the operational mode to be specified for the SSID (for example, as the primary wireless device or the secondary wireless device) may overwrite the current setting in the case of an overlap of the SSID.

In the case that an overlap is detected as a result of the check or the overwriting is not selected by the setter in the subsequent check procedure, the overwriting is not performed.

In the case no overlap is detected as a result of the check or the overwriting is selected by the setter in the subsequent check procedure, the overwriting is performed.

In this manner, the same SSID is not selected for multiple settings. Thus the connection to an unintended device can be reduced.

Before an SSID is assigned to the radiographic device 3, the overlap of this SSID with another SSID used during a specific period in the past may be checked.

The past use of the SSID may be determined by, for example, the log of assignment of the SSID or of the access to the SSID.

For example, the console 2 may have a function that displays a list of the SSIDs assigned or accessed in the past and deletes the SSID specified by the user from the list.

In this manner, the selection of the SSID used in the past but not at present can be managed.

### Example 2-3: Display of Mode for Primary Device or Secondary Device

In the radiography using the radiographic device 3 operable as both the primary wireless device and the secondary wireless device, the user cannot know whether the radiographic device 3 is currently defined as the primary wireless device or the secondary wireless device and may capture an image in an unintended operational mode.

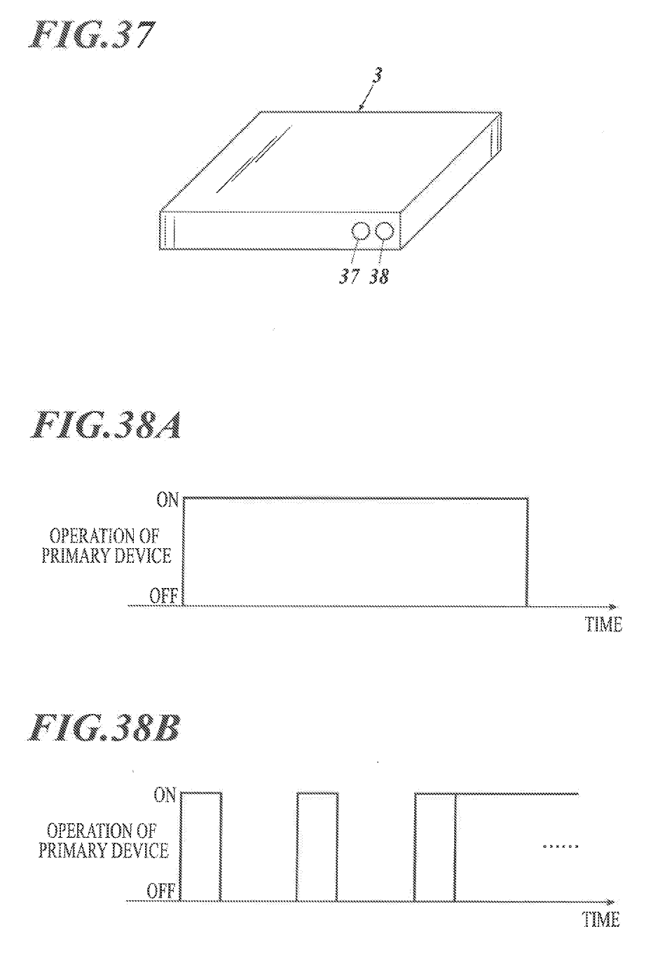

To solve this problem, a mode display 37 may be provided on the radiographic device 3 to display the current mode (the mode for operation as the primary wireless device or the secondary wireless device) as illustrated in FIG. 37.

Specifically, characters are displayed or colors are modified for notification of the mode.

In this manner, the user can know the current mode. Thus, the user can prevent an image from being captured in a mode inconsistent with the intention of the user.

### Example 2-4: Switching Between Primary Device and Secondary Device with Button

In the example described above, the radiographic device 3 operable as both the primary wireless device and the secondary wireless device must be inserted into the cradle for switching of the modes, which operation is time-consuming.

This is particularly crucial in the emergency medical services.

To solve such a problem, a switching button 38 operable by the user may be provided on the radiographic device 3 as illustrated in FIG. 37. The user operates the switching button 38 to switch the mode from the primary wireless device to the secondary wireless device, that is, the user presses the switching button 38 to change the mode to the secondary wireless device.

In this manner, the mode can be readily switched between the primary wireless device and the secondary wireless device.

### Example 2-5: Approach to Reduction of Power Consumption

As illustrated in FIG. 38A, the radiographic device 3 operating as the primary wireless device continues to operate or communicate while the power is on. Thus, the battery of the radiographic device 3 as the primary wireless device is exhausted before the battery of the radiographic device 3 as the secondary wireless device is exhausted.

To solve such a problem, the radiographic device 3 as the primary wireless device may intermittently operate in the absence of signals from the secondary wireless device or may continuously operate as the primary wireless device while the radiographic device 3 is receiving signals from the secondary wireless device, as illustrated in FIG. 38B.

If the non-operating time as the primary wireless device does not reach a predetermined value during the intermittent operation of the radiographic device 3 as the primary wireless device, the network connection diagram continues to display the radiographic device 3. This can prevent the user from misunderstanding that the connection between the console 2 and the radiographic device 3 is lost.

Such a manner can shorten the time of operation as the primary wireless device during a predetermined period and reduce the power consumption, resulting in an extended lifetime of the battery of the radiographic device 3 operating as the primary wireless device.

### Example 3-1: Embodiment (1) Using Visiting Cart

A traditional visiting cart 100A including, for example, an irradiator 1, a console 2, a power source 100a, and a radiographic device 3A requires an in-house primary wireless device (AP 7A) connected to, for example, an external wireless communication interface 100b via a network. Such a primary wireless device may be unavailable in the radiography at any site, for example, a sickroom or an operating room, other than the laboratory.

Before or during the movement of the visiting cart for capturing of a radiographic image, the information (name, gender, age, radiographic portion, and radiographic scheme) on a target patient should be acquired. Thus, the radiographic device 3A should function as the secondary wireless device that acquires the information on the patient from the in-house AP 7A operating as the primary wireless device.

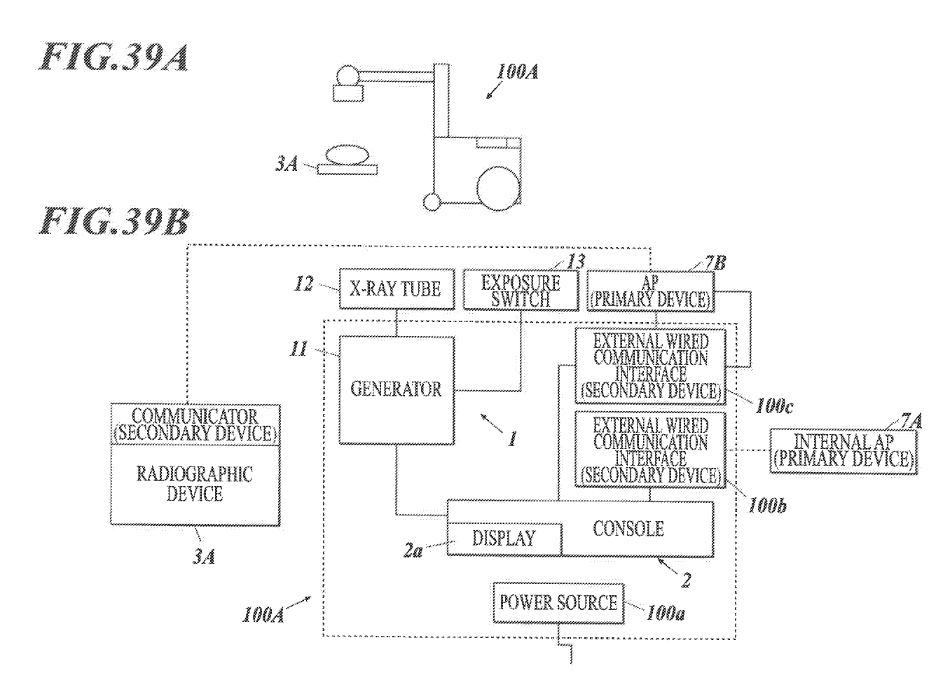

For this reason, the traditional visiting cart 100A in FIG. 39A requires an additional AP 7B operating as a primary wireless device disposed outside the housing of the visiting cart 100A and connected to an external wired communication interface 100c, as illustrated in FIG. 39B. It should be noted that the power cabling is not illustrated in FIGS. 39B and 40.

Meanwhile, the radiographic system 100 according to the example including a visiting cart 100 in FIGS. 40A and 40B requires no AP on the visiting cart 100 and enables the radiographic device 3 to be directly connected to the console 2 via a wireless communicator of the visiting cart 100; the radiographic device 3 operates as the primary wireless device.

The on-cart AP, which is disposed outside the housing of the visiting cart in many cases, may be hit and broken, resulting in a communication failure. In contrast, the configuration according to the present example can prevent the communication failure or reduce the probability of the communication failure, resulting in a stable operation of the visiting cart.

The visiting cart is operated by a mounted battery having a limited power capacity. The power consumption by the AP should be kept low for a longer operation of the cart. The absence of the on-cart AP can reduce the power consumption and enables the visiting cart to operate longer.

The lack of the additional AP can also reduce the production cost of the visiting cart.

Since the on-cart AP is not required, no cable extends from the visiting cart, resulting in elimination of issues associated with cabling, such as entanglement.

### Example 3-2: Embodiment (2) Using Visiting Cart

The communication target or the primary wireless device to communicate with the wireless interface of the visiting cart can be selected depending on the state of the visiting cart capturing an image or the state of the visiting cart during movement.

For example, in the case that the arm of the visiting cart is stored in a predetermined storage site, the AP (primary wireless device) in the internal network is preferentially selected and connected to the wireless interface of the visiting cart.

In contrast, in the case that the arm of the visiting cart is away from the storage site or the electromagnetic brake of the visiting cart is actuated for stop of the wheel, the radiographic device 3 operating as the primary wireless device is preferentially selected and connected to the wireless interface of the visiting cart.

The communication target may be selected after elapse of a predetermined time while the visiting cart is in any of the above states.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese patent application No. 2018-009975, filed on Jan. 24, 2018, is incorporated herein by reference in its entirety.

What is claimed is:

1. A radiographic communication system comprising:
at least one radiographic device that receives radiation to generate data of a radiographic image and that operates as a primary device in wireless communication; and
an information processor that operates as a secondary device in the wireless communication,
wherein the information processor comprises:
a memory that stores combination information including a communication identifier and an address of the radiographic device;
at least one operation interface that is associated with the combination information and is operated by a user;
a communicator; and
a hardware processor that uses the communicator to establish communication with the radiographic device based on the communication identifier in the combination information in response to an operation of the operation interface, the hardware processor performing at least one of transmission and reception of data to and from the radiographic device via the communicator based on the address in the combination information.

2. The radiographic communication system according to claim 1, wherein
the at least one radiographic device comprises multiple radiographic devices having respective communication identifiers and addresses, and
the information processor comprises multiple operation interfaces for respective pieces of combination information including communication identifiers and addresses of the radiographic devices, the information processor storing the pieces of combination information in the memory, and the information processor specifying one of the pieces of combination information in response to an operation of any of the operation interfaces.

3. A radiographic communication system comprising:
a radiographic device that receives radiation to generate data of a radiographic image and that operates as a primary device in wireless communication; and
an information processor that operates as a secondary device in the wireless communication,
wherein the information processor comprises:
a memory that stores combination information including a communication identifier and an address of the radiographic device, the communication identifier and the address being associated with a predetermined radiographic condition;
an operation interface that is associated with the radiographic condition and is operated by a user;
a communicator; and
a hardware processor that uses the communicator to establish communication with the radiographic device based on the communication identifier in the combination information associated with the radiographic condition selected in response to an operation of the operation interface, the hardware processor performing at least one of transmission and reception of data to and from the radiographic device based on the address in the combination information associated with the selected radiographic condition.

4. The radiographic communication system according to claim 3, wherein the radiographic condition comprises any one of a radiographic portion, a radiographic location, a radiographic room, a radiographic direction, and the type of the radiographic device.

5. The radiographic communication system according to claim 1, wherein the communication identifier comprises an identifier of the communicator for the wireless communication.

6. The radiographic communication system according to claim 1, wherein the communication identifier comprises a combination of an identifier and an access key for the wireless network.

7. The radiographic communication system according to claim 1, wherein the address is one of a MAC address, an IP address, and a host name.

8. A radiographic system comprising:
an irradiator that generates radiation; and
the radiographic communication system according to claim 1.
